(19) United States
(12) Patent Application Publication
(10) Pub. No.: US 2005/0239089 A1
(43) Pub. Date: Oct. 27, 2005

(54) Mobility cassettes

(76) Inventors: Martin D. Johnson, Woodside, CA (US); Michael W. Hunkapiller, San Carlos, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/861,314

(22) Filed: Jun. 4, 2004

Related U.S. Application Data
(60) Provisional application No. 60/476,434, filed on Jun. 6, 2003.

(51) Int. Cl. C12Q 1/68

(52) U.S. Cl. 435/6

(57) ABSTRACT

Mobility cassettes are provided. Methods of using mobility cassettes for the detection of nucleic acids are provided.

This application claims the benefit of priority of U.S. Provisional Application No. 60/476,434, filed Jun. 6, 2003, which is incorporated by reference herein in its entirety for any purpose.

### I. FIELD OF THE INVENTION

The invention relates to methods and compositions for the analysis of nucleic acids.

### II. BACKGROUND

The detection of the presence or absence of one or more target sequences in a sample containing one or more target sequences is commonly practiced. For example, the detection of cancer and many infectious diseases, such as AIDS and hepatitis, routinely includes screening biological samples for the presence or absence of diagnostic nucleic acid sequences. Also, detecting the presence or absence of nucleic acid sequences is often used in forensic science, paternity testing, genetic counseling, and organ transplantation.

### III. SUMMARY OF THE INVENTION

In certain embodiments, methods are provided for detecting at least one analyte polynucleotide comprising a tag sequence. In certain embodiments, the methods comprise forming a ligation reaction composition comprising the at least one analyte polynucleotide and at least one mobility cassette. In certain embodiments, the at least one mobility cassette comprises a first nucleic acid strand comprising a mobility modifier, and a second nucleic acid strand comprising a tag complement sequence that is complementary to the tag sequence of the analyte polynucleotide. In certain embodiments, a portion of the second nucleic acid strand is hybridized to the first nucleic acid strand and at least a portion of the tag complement sequence is not hybridized to the first nucleic acid strand. In certain embodiments, the first nucleic acid strand is suitable for ligation to the analyte polynucleotide when the first nucleic acid strand and the analyte polynucleotide are hybridized to one another.

In certain embodiments, the methods further comprise subjecting the ligation reaction composition to at least one cycle of ligation, wherein the first nucleic acid strand and the analyte polynucleotide are ligated to form a mobility modifier ligation product comprising the first nucleic acid strand and the analyte polynucleotide. In certain embodiments, the methods further comprise detecting the at least one analyte polynucleotide by analyzing the mobility modifier ligation product using a mobility-dependent analysis technique.

In certain embodiments, methods are provided for detecting at least one primer extension product. In certain embodiments, the methods comprise forming an extension reaction composition comprising a polymerase, at least one target nucleic acid sequence, and a primer for each target nucleic acid sequence, the primer comprising a target-specific portion and a tag sequence. In certain embodiments, the methods further comprise incubating the extension reaction composition under appropriate conditions to generate a primer extension product for each target nucleic acid, the primer extension product comprising the tag sequence. In certain embodiments, the methods further comprise forming a ligation reaction composition comprising the primer extension product for each target nucleic acid and a mobility cassette for each target nucleic acid. In certain embodiments, the mobility cassette comprises a first nucleic acid strand comprising a mobility modifier, and a second nucleic acid strand comprising a complementary tag sequence that is complementary to the tag sequence of the primer extension product. In certain embodiments, a portion of the second nucleic acid strand is hybridized to the first nucleic acid strand and at least a portion of the complementary tag sequence is not hybridized to the first nucleic acid strand. In certain embodiments, the first nucleic acid strand is suitable for ligation to the primer extension product when the first nucleic acid strand and the primer extension product are hybridized adjacent to one another on the second nucleic acid strand. In certain embodiments, the methods further comprise subjecting the ligation reaction composition to at least one cycle of ligation, wherein the first nucleic acid strand and the primer extension product for each target nucleic acid that are adjacently hybridized on the second nucleic acid strand for each target nucleic acid are ligated to form a mobility modifier ligation product comprising the first nucleic acid strand and the primer extension product. In certain embodiments, the methods further comprise detecting the primer extension product for each target nucleic acid sequence by analyzing the mobility modifier ligation product or a portion of the mobility modifier ligation product for each primer extension product using a mobility-dependent analysis technique.

In certain embodiments, methods are provided for detecting at least one target nucleic acid sequence in a sample. In certain embodiments, the methods comprise forming a first ligation reaction composition comprising the sample and a probe set for each target nucleic acid sequence, the probe set comprising (i) at least one first probe, comprising a first target-specific portion, and (ii) at least one second probe, comprising a second target-specific portion. In certain embodiments, the probes in each set are suitable for ligation together when hybridized adjacent to one another on the at least one target nucleic acid sequence, and at least one probe in each probe set further comprises at least one tag sequence. In certain embodiments, the methods further comprise subjecting the first ligation reaction composition to at least one cycle of ligation, wherein adjacently hybridized probes are ligated to form a first ligation product comprising the first and second target-specific portions and the tag sequence.

In certain embodiments, the methods further comprise forming a second ligation reaction composition comprising the first ligation product and a mobility cassette. In certain embodiments, the mobility cassette comprises a first nucleic acid strand comprising a mobility modifier, and a second nucleic acid strand comprising a complementary tag sequence that is complementary to the tag sequence of the first ligation product. In certain embodiments, a portion of the second nucleic acid strand is hybridized to the first nucleic acid strand and wherein at least a portion of the complementary tag sequence is not hybridized to the first nucleic acid strand, and the first nucleic acid strand is suitable for ligation to the first ligation product when the first nucleic acid strand and the first ligation product are hybridized adjacent to one another on the second nucleic acid strand.

In certain embodiments, the methods further comprise subjecting the second ligation reaction composition to at least one cycle of ligation, wherein the first nucleic acid strand and the first ligation product that are adjacently hybridized on the second nucleic acid strand are ligated to form a mobility modifier ligation product comprising the first nucleic acid strand and the first ligation product. In certain embodiments, the methods further comprise detecting the at least one target nucleic acid sequence by analyzing the mobility modifier ligation product or a portion of the mobility modifier ligation product using a mobility-dependent analysis technique.

In certain embodiments, methods are provided for detecting at least one target nucleic acid sequence in a sample. In certain embodiments, the methods comprise forming a first ligation reaction composition comprising: the sample and a probe set for each target nucleic acid sequence. In certain embodiments, the probe set comprises (a) at least one first probe, comprising a first target-specific portion, a tag sequence, and a 5′ primer-specific portion, wherein the 5′ primer-specific portion comprises a sequence, and wherein the tag sequence is located between the 5′ primer-specific portion and the first target-specific portion, and (b) at least one second probe, comprising a second target-specific portion and a 3′ primer-specific portion, wherein the 3′ primer-specific portion comprises a sequence, wherein the probes in each set are suitable for ligation together when hybridized adjacent to one another on the at least one target nucleic acid sequence.

In certain embodiments, the methods further comprise subjecting the ligation reaction composition to at least one cycle of ligation, wherein adjacently hybridized probes are ligated to form a first ligation product comprising the first and second target-specific portions, the tag sequence, and the 3′ and 5′ primer-specific portions.

In certain embodiments, the methods further comprise forming a first amplification reaction comprising a DNA polymerase, the first ligation product, and at least one primer set comprising at least one first primer comprising the sequence of the 5′ primer-specific portion, and at least one second primer comprising a sequence complementary to the sequence of the 3′ primer-specific portion of the ligation product.

In certain embodiments, the methods further comprise subjecting the first amplification reaction composition to at least one cycle of amplification to generate an amplification product.

In certain embodiments, the methods further comprise forming a second ligation reaction composition comprising the amplification product and a mobility cassette. In certain embodiments, the mobility cassette comprises: a first nucleic acid strand comprising a mobility modifier; and a second nucleic acid strand comprising a complementary tag sequence that is complementary to the 5′ primer-specific portion and the tag sequence of the amplification product, wherein a portion of the second nucleic acid strand is hybridized to the first nucleic acid strand and wherein at least a portion of the complementary tag sequence is not hybridized to the first nucleic acid strand. In certain embodiments, the first nucleic acid strand is suitable for ligation to the amplification product when the first nucleic acid strand and the amplification product are hybridized adjacent to one another on the second nucleic acid strand.

In certain embodiments, the methods further comprise subjecting the second ligation reaction composition to at least one cycle of ligation, wherein the first nucleic acid strand and the amplification product that are adjacently hybridized on the second nucleic acid strand are ligated to form a mobility modifier ligation product comprising the first nucleic acid strand and the amplification product. In certain embodiments, the methods further comprise detecting the at least one target nucleic acid sequence by analyzing the mobility modifier ligation product or a portion of the mobility modifier ligation product using a mobility-dependent analysis technique.

In certain embodiments, methods are provided for detecting at least one target nucleic acid sequence in a sample. In certain embodiments, the methods comprise forming a first ligation reaction composition comprising: the sample and a probe set for each target nucleic acid sequence, the probe set comprising (a) at least one first probe, comprising a first target-specific portion and a 5′ primer-specific portion, wherein the 5′ primer-specific portion comprises a sequence, and (b) at least one second probe, comprising a second target-specific portion, a tag sequence, and a 3′ primer-specific portion, wherein the 3′ primer-specific portion comprises a sequence, and wherein the tag sequence is located between the 3′ primer-specific portion and the second target-specific portion. In certain embodiments, the probes in each set are suitable for ligation together when hybridized adjacent to one another on the at least one target nucleic acid sequence.

In certain embodiments, the methods further comprise subjecting the first ligation reaction composition to at least one cycle of ligation, wherein adjacently hybridized probes are ligated to form a first ligation product comprising the first and second target-specific portions, the tag sequence, and the 3′ and 5′ primer-specific portions.

In certain embodiments, the methods further comprise forming a first amplification reaction composition comprising a DNA polymerase, the first ligation product, and at least one primer set comprising at least one first primer comprising the sequence of the 5′ primer specific portion, and at least one second primer comprising a sequence complementary to the sequence of the 3′ primer-specific portion.

In certain embodiments, the methods further comprise subjecting the first amplification reaction composition to at least one cycle of amplification to generate an amplification product.

In certain embodiments, the methods further comprise forming a second ligation reaction composition comprising the amplification product and a mobility cassette, wherein the mobility cassette comprises a first nucleic acid strand comprising a mobility modifier; and a second nucleic acid strand comprising a complementary tag sequence that is complementary to the 3′ primer-specific portion and the tag sequence of the amplification product, wherein a portion of the second nucleic acid strand is hybridized to the first nucleic acid strand and wherein at least a portion of the complementary tag sequence is not hybridized to the first nucleic acid strand. In certain embodiments, the first nucleic acid strand is suitable for ligation to the amplification product when the first nucleic acid strand and the amplification product are hybridized adjacent to one another on the second nucleic acid strand.

In certain embodiments, the methods further comprise subjecting the second ligation reaction composition to at least one cycle of ligation, wherein the first nucleic acid strand and the amplification product that are adjacently hybridized on the second nucleic acid strand are ligated to form a mobility modifier ligation product comprising the first nucleic acid strand and the amplification product; and detecting the at least one target nucleic acid sequence by analyzing the mobility modifier ligation product or a portion of the mobility modifier ligation product using a mobility-dependent analysis technique.

In certain embodiments, a mobility cassette is provided. In certain embodiments, the mobility cassette comprises a first nucleic acid strand comprising a mobility modifier; and a second nucleic acid strand comprising a complementary tag sequence that is complementary to a tag sequence of a target product. In certain embodiments, a portion of the second nucleic acid strand is hybridized to the first nucleic acid strand and at least a portion of the complementary tag sequence is not hybridized to the first nucleic acid strand. In certain embodiments, the first nucleic acid strand is suitable for ligation to the target product when the first nucleic acid strand and the target product are hybridized adjacent to one another on the second nucleic acid strand. In certain embodiments, the mobility modifier is a polyethylene oxide molecule. In certain embodiments, the mobility modifier is a polynucleotide.

In certain embodiments, kits for detecting at least one target nucleic acid sequence are provided. In certain embodiments, the kits comprise (a) at least one of (i) a probe comprising a tag sequence and a target-specific portion that can hybridize to a target nucleic acid sequence; and (ii) a primer comprising a tag sequence and a target-specific portion that can hybridize to a target nucleic acid sequence; and (b) a mobility cassette comprising; a first nucleic acid strand comprising a mobility modifier; and a second nucleic acid strand comprising a complementary tag sequence that is complementary to the tag sequence. In certain embodiments, a portion of the second nucleic acid strand is hybridized to the first nucleic acid strand and at least a portion of the complementary tag sequence is not hybridized to the first nucleic acid strand. In certain embodiments, the first nucleic acid strand is suitable for ligation to the tag sequence when the first nucleic acid strand and the tag sequence are hybridized adjacent to one another on the second nucleic acid strand.

### IV. BRIEF DESCRIPTION OF THE FIGURES

FIG. 1 illustrates certain embodiments of a mobility cassette comprising a mobility modifier.

FIG. 2 illustrates certain embodiments of a method of detecting an analyte comprising a tag sequence using a mobility modifier cassette comprising a mobility modifier and subsequent detection using a mobility-dependent analysis technique.

FIG. 3 illustrates certain embodiments of hybridization of a target polynucleotide to a mobility cassette, and subsequent detection by mobility-dependent analysis (e.g., electrophoresis).

FIG. 4 illustrates certain embodiments of generating a labeled primer extension product with a unique tag sequence, and subsequent detection by mobility-dependent analysis (e.g., electrophoresis).

FIG. 5 illustrates certain embodiments of detecting a target polynucleotide by detecting a Taqman™ cleavage product with a mobility cassette.

FIG. 6 illustrates certain embodiments of detecting a target polynucleotide by detecting a cleavage product from a cleavage assay technique (e.g., the Invader™ assay), and subsequent detection using a mobility cassette and a mobility-dependent analysis technique.

FIG. 7 illustrates certain embodiments of probe pairs comprising a first probe and a second probe that, when hybridized to a complementary target, may ligate together.

FIG. 8 illustrates certain embodiments of detecting a polynucleotide using an oligonucleotide ligation assay and PCR to generate nucleic acid for hybridization to mobility modifier cassettes.

FIG. 9 illustrates certain embodiments of detecting a polynucleotide using an oligonucleotide ligation assay to generate nucleic acids for hybridization to mobility modifier cassettes.

FIG. 10 illustrates certain embodiments of detecting a polynucleotide using an oligonucleotide ligation assay and PCR to generate nucleic acids for hybridization to mobility modifier cassettes.

FIG. 11 illustrates certain embodiments of detecting a polynucleotide using an oligonucleotide ligation assay to generate nucleic acids for hybridization to mobility modifier cassettes.

### V. DETAILED DESCRIPTION OF CERTAIN EXEMPLARY EMBODIMENTS

It is to be understood that both the foregoing general description and the following detailed description are exemplary and explanatory only and are not restrictive of the invention, as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. U.S. Patent Application Ser. No. 60/445,494, filed Feb. 7, 2003 is incorporated by reference herein in its entirety for any purpose.

A. Certain Definitions

The term “nucleotide base”, as used herein, refers to a substituted or unsubstituted aromatic ring or rings. In certain embodiments, the aromatic ring or rings contain at least one nitrogen atom. In certain embodiments, the nucleotide base is capable of forming Watson-Crick and/or Hoogsteen hydrogen bonds with an appropriately complementary nucleotide base. Exemplary nucleotide bases and analogs thereof include, but are not limited to, naturally occurring nucleotide bases adenine, guanine, cytosine, uracil, thymine, and analogs of the naturally occurring nucleotide bases, e.g., 7-deazaadenine, 7-deazaguanine, 7-deaza-8-azaguanine, 7-deaza-8-azaadenine, N6-Δ2-isopentenyladenine (6iA), N6-Δ2-isopentenyl-2-methylthioadenine (2 ms6iA), N2-dimethylguanine (dmG), 7-methylguanine (7mG), inosine, nebularine, 2-aminopurine, 2-amino-6-chloropurine, 2,6-diaminopurine, hypoxanthine, pseudouridine, pseudocytosine, pseudo isocytosine, 5-propynylcytosine, isocytosine, isoguanine, 7-deazaguanine, 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, pyrazolo[3,4-D]pyrimidines (see, e.g., U.S. Pat. Nos. 6,143,877 and 6,127,121 and PCT published application WO 01/38584), ethenoadenine, indoles such as nitroindole and 4-methylindole, and pyrroles such as nitropyrrole. Certain exemplary nucleotide bases can be found, e.g., in Fasman, 1989, Practical Handbook of Biochemistry and Molecular Biology, pp. 385-394, CRC Press, Boca Raton, Fla., and the references cited therein.

The term “nucleotide,” as used herein, refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR₂ or halogen groups, where each R is independently H, C₁-C₆ alkyl or C₅-C₁₄ aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′- and 3′-4′-linked and other “locked” or “LNA”, bicyclic sugar modifications (see, e.g., PCT published application nos. WO 98/22489, WO 98/39352; and WO 99/14226). Exemplary LNA sugar analogs within a polynucleotide include, but are not limited to, the structures: 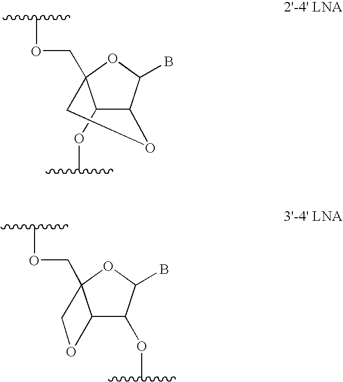
where B is any nucleotide base.

Modifications at the 2′- or 3′-position of ribose include, but are not limited to, hydrogen, hydroxy, methoxy, ethoxy, allyloxy, isopropoxy, butoxy, isobutoxy, methoxyethyl, alkoxy, phenoxy, azido, amino, alkylamino, fluoro, chloro and bromo. Nucleotides include, but are not limited to, the natural D optical isomer, as well as the L optical isomer forms (see, e.g., Garbesi (1993) Nucl. Acids Res. 21:4159-65; Fujimori (1990) J. Amer. Chem. Soc. 112:7435; Urata, (1993) Nucleic Acids Symposium Ser. No. 29:69-70). When the nucleotide base is purine, e.g. A or G, the ribose sugar is attached to the N⁹-position of the nucleotide base. When the nucleotide base is pyrimidine, e.g. C, T or U, the pentose sugar is attached to the N¹-position of the nucleotide base, except for pseudouridines, in which the pentose sugar is attached to the C5 position of the uracil nucleotide base (see, e.g., Kornberg and Baker, (1992) DNA Replication, 2^{nd} Ed., Freeman, San Francisco, Calif.).

One or more of the pentose carbons of a nucleotide may be substituted with a phosphate ester having the formula: 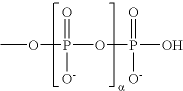
where α is an integer from 0 to 4. In certain embodiments, α is 2 and the phosphate ester is attached to the 3′- or 5′-carbon of the pentose. In certain embodiments, the nucleotides are those in which the nucleotide base is a purine, a 7-deazapurine, a pyrimidine, or an analog thereof. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group may include sulfur substitutions for the various oxygens, e.g. α-thio-nucleotide 5′-triphosphates. For a review of nucleotide chemistry, see: Shabarova, Z. and Bogdanov, A. Advanced Organic Chemistry of Nucleic Acids, VCH, New York, 1994.

The term “nucleotide analog,” as used herein, refers to embodiments in which the pentose sugar and/or the nucleotide base and/or one or more of the phosphate esters of a nucleotide may be replaced with its respective analog. In certain embodiments, exemplary pentose sugar analogs are those described above. In certain embodiments, the nucleotide analogs have a nucleotide base analog as described above. In certain embodiments, exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., and may include associated counterions.

Also included within the definition of “nucleotide analog” are nucleotide analog monomers which can be polymerized into polynucleotide analogs in which the DNA/RNA phosphate ester and/or sugar phosphate ester backbone is replaced with a different type of internucleotide linkage. Exemplary polynucleotide analogs include, but are not limited to, peptide nucleic acids, in which the sugar phosphate backbone of the polynucleotide is replaced by a peptide backbone.

As used herein, the terms “polynucleotide,” “oligonucleotide,” and “nucleic acid” are used interchangeably and mean single-stranded and double-stranded polymers of nucleotide monomers, including 2′-deoxyribonucleotides (DNA) and ribonucleotides (RNA) linked by internucleotide phosphodiester bond linkages, or internucleotide analogs, and associated counter ions, e.g., H⁺, NH₄⁺, trialkylammonium, Mg²⁺, Na⁺ and the like. A nucleic acid may be composed entirely of deoxyribonucleotides, entirely of ribonucleotides, or chimeric mixtures thereof. The nucleotide monomer units may comprise any of the nucleotides described herein, including, but not limited to, naturally occurring nucleotides and nucleotide analogs nucleic acids typically range in size from a few monomeric units, e.g. 5-40 when they are sometimes referred to in the art as oligonucleotides, to several thousands of monomeric nucleotide units. Unless denoted otherwise, whenever a nucleic acid sequence is represented, it will be understood that the nucleotides are in 5′ to 3′ order from left to right and that “A” denotes deoxyadenosine or an analog thereof, “C” denotes deoxycytidine or an analog thereof, “G” denotes deoxyguanosine or an analog thereof, and “T” denotes thymidine or an analog thereof, unless otherwise noted.

Nucleic acids include, but are not limited to, genomic DNA, cDNA, hnRNA, mRNA, rRNA, tRNA, fragmented nucleic acid, nucleic acid obtained from subcellular organelles such as mitochondria or chloroplasts, and nucleic acid obtained from microorganisms or DNA or RNA viruses that may be present on or in a biological sample.

Nucleic acids may be composed of a single type of sugar moiety, e.g., as in the case of RNA and DNA, or mixtures of different sugar moieties, e.g., as in the case of RNA/DNA chimeras. In certain embodiments, nucleic acids are ribopolynucleotides and 2′-deoxyribopolynucleotides according to the structural formulae below: 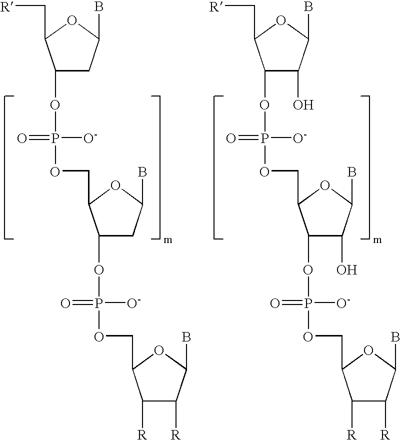
wherein each B is independently the base moiety of a nucleotide, e.g., a purine, a 7-deazapurine, a pyrimidine, or an analog nucleotide; each m defines the length of the respective nucleic acid and can range from zero to thousands, tens of thousands, or even more; each R is independently selected from the group comprising hydrogen, halogen, —R″, —OR″, and —NR″R″, where each R″ is independently (C1-C6) alkyl or (C5-C14) aryl, or two adjacent Rs are taken together to form a bond such that the ribose sugar is 2′,3′-didehydroribose; and each R′ is independently hydroxyl or 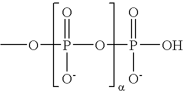
where α is zero, one or two.

In certain embodiments of the ribopolynucleotides and 2′-deoxyribopolynucleotides illustrated above, the nucleotide bases B are covalently attached to the C1′ carbon of the sugar moiety as previously described.

The terms “nucleic acid,” “polynucleotide,” and “oligonucleotide” may also include nucleic acid analogs, polynucleotide analogs, and oligonucleotide analogs. The terms “nucleic acid analog,” “polynucleotide analog,” and “oligonucleotide analog” are used interchangeably and, as used herein, refer to a nucleic acid that contains at least one nucleotide analog and/or at least one phosphate ester analog and/or at least one pentose sugar analog. Also included within the definition of nucleic acid analogs are nucleic acids in which the phosphate ester and/or sugar phosphate ester linkages are replaced with other types of linkages, such as N-(2-aminoethyl)-glycine amides and other amides (see, e.g., Nielsen et al., 1991, Science 254: 1497-1500; WO 92/20702; U.S. Pat. No. 5,719,262; U.S. Pat. No. 5,698,685;); morpholinos (see, e.g., U.S. Pat. No. 5,698,685; U.S. Pat. No. 5,378,841; U.S. Pat. No. 5,185,144); carbamates (see, e.g., Stirchak & Summerton, 1987, J. Org. Chem. 52: 4202); methylene(methylimino) (see, e.g., Vasseur et al., 1992, J. Am. Chem. Soc. 114: 4006); 3′-thioformacetals (see, e.g., Jones et al., 1993, J. Org. Chem. 58: 2983); sulfamates (see, e.g., U.S. Pat. No. 5,470,967); 2-aminoethylglycine, commonly referred to as PNA (see, e.g., Buchardt, WO 92/20702; Nielsen (1991) Science 254:1497-1500); and others (see, e.g., U.S. Pat. No. 5,817,781; Frier & Altman, 1997, Nucl. Acids Res. 25:4429 and the references cited therein). Phosphate ester analogs include, but are not limited to, (i) C₁-C₄ alkylphosphonate, e.g. methylphosphonate; (ii) phosphoramidate; (iii) C₁-C₆ alkyl-phosphotriester; (iv) phosphorothioate; and (v) phosphorodithioate.

The terms “analyte” and “analyte polynucleotide,” refer to a nucleotide sequence that becomes bound to a mobility modifier cassette and is detected. An analyte may be identified by its unique sequence.

The term “target” and “target nucleic acid sequence” refer to a nucleic acid sequence to be detected in a sample. In certain embodiments, the target nucleic acid sequence is detected directly with the mobility modifier cassette, and the target nucleic acid sequence is thus an analyte polynucleotide. In certain embodiments, the term “target nucleic acid sequence” refers to a polynucleotide, the presence or absence of which is being tested, while the term “analyte polynucleotide” refers to a polynucleotide that is generated only in the presence of the target nucleic acid sequence. The presence or absence of the analyte polynucleotide is tested in order to determine the presence or absence of the target nucleic acid sequence in the sample.

The person of ordinary skill will appreciate that while an analyte polynucleotide may be a single-stranded molecule, the opposing strand of a double-stranded molecule comprises a complementary sequence that may also be used as an analyte polynucleotide.

The terms “annealing” and “hybridization” are used interchangeably and mean the base-pairing interaction of one nucleic acid with another nucleic acid that results in formation of a duplex, triplex, or other higher-ordered structure. In certain embodiments, the primary interaction is base specific, e.g., A/T and G/C, by Watson/Crick and Hoogsteen-type hydrogen bonding. In certain embodiments, base-stacking and hydrophobic interactions may also contribute to duplex stability.

An “enzymatically active mutant or variant thereof,” when used in reference to an enzyme such as a polymerase or a ligase, means a protein with appropriate enzymatic activity. Thus, for example, but without limitation, an enzymatically active mutant or variant of a DNA polymerase is a protein that is able to catalyze the stepwise addition of appropriate deoxynucleoside triphosphates into a nascent DNA strand in a template-dependent manner. An enzymatically active mutant or variant differs from the “generally-accepted” or consensus sequence for that enzyme by at least one amino acid, including, but not limited to, substitutions of one or more amino acids, addition of one or more amino acids, deletion of one or more amino acids, and alterations to the amino acids themselves. With the change, however, at least some catalytic activity is retained. In certain embodiments, the changes involve conservative amino acid substitutions. Conservative amino acid substitution may involve replacing one amino acid with another that has, e.g., similar hydorphobicity, hydrophilicity, charge, or aromaticity. In certain embodiments, conservative amino acid substitutions may be made on the basis of similar hydropathic indices. A hydropathic index takes into account the hydrophobicity and charge characteristics of an amino acid, and in certain embodiments, may be used as a guide for selecting conservative amino acid substitutions. The hydropathic index is discussed, e.g., in Kyte et al., J. Mol. Biol., 157:105-131 (1982). It is understood in the art that conservative amino acid substitutions may be made on the basis of any of the aforementioned characteristics.

Alterations to the amino acids may include, but are not limited to, glycosylation, methylation, phosphorylation, biotinylation, and any covalent and noncovalent additions to a protein that do not result in a change in amino acid sequence. “Amino acid” as used herein refers to any amino acid, natural or normatural, that may be incorporated, either enzymatically or synthetically, into a polypeptide or protein.

Fragments, for example, but without limitation, proteolytic cleavage products, are also encompassed by this term, provided that at least some enzyme catalytic activity is retained.

The skilled artisan will readily be able to measure catalytic activity using an appropriate well-known assay. Thus, an appropriate assay for polymerase catalytic activity might include, for example, measuring the ability of a variant to incorporate, under appropriate conditions, rNTPs or dNTPs into a nascent polynucleotide strand in a template-dependent manner. Likewise, an appropriate assay for ligase catalytic activity might include, for example, the ability to ligate adjacently hybridized oligonucleotides comprising appropriate reactive groups. Protocols for such assays may be found, among other places, in Sambrook et al., Molecular Cloning, A Laboratory Manual, Cold Spring Harbor Press (1989) (hereinafter “Sambrook et al.”), Sambrook and Russell, Molecular Cloning, Third Edition, Cold Spring Harbor Press (2000) (hereinafter “Sambrook and Russell”), Ausbel et al., Current Protocols in Molecular Biology (1993) including supplements through April 2001, John Wiley & Sons (hereinafter “Ausbel et al.”).

The terms “tag” and “tag complement,” as used herein, refer to single-stranded nucleic acids that complement another single-stranded nucleic acid. In certain embodiments, the term “tag complement” refers to a nucleic acid that is complementary to the nucleic acid designated as the “tag.”

“Probes”, according to the present invention, comprise oligonucleotides that comprise a specific portion that is designed to hybridize in a sequence-specific manner with a complementary region on a specific nucleic acid sequence, e.g., a target nucleic acid sequence. In certain embodiments, the specific portion of the probe may be specific for a particular sequence, or alternatively, may be degenerate, e.g., specific for a set of sequences.

A “probe set” according to the present invention is a group of two or more probes designed to detect at least one target. As a non-limiting example, a probe set may comprise two nucleic acid probes designed to hybridize to a target such that, when the two probes are hybridized to the target adjacent to one another, they are suitable for ligation together.

When used in the context of the present invention, “suitable for ligation” refers to at least one first target-specific probe and at least one second target-specific probe, each comprising an appropriately reactive group. Exemplary reactive groups include, but are not limited to, a free hydroxyl group on the 3′ end of the first probe and a free phosphate group on the 5′ end of the second probe. Exemplary pairs of reactive groups include, but are not limited to: phosphorothioate and tosylate or iodide; esters and hydrazide; RC(O)S—, haloalkyl, or RCH₂S and α-haloacyl; thiophosphoryl and bromoacetoamido groups. Exemplary reactive groups include, but are not limited to, S-pivaloyloxymethyl-4-thiothymidine. Additionally, in certain embodiments, first and second target-specific probes are hybridized to the target sequence such that the 3′ end of the first target-specific probe and the 5′ end of the second target-specific probe are immediately adjacent to allow ligation.

In this application, a statement that one sequence is the same as or is complementary to another sequence encompasses situations where both of the sequences are completely the same or complementary to one another, and situations where only a portion of one of the sequences is the same as, or is complementary to, a portion or the entire other sequence. Here, the term “sequence” encompasses, but is not limited to, nucleic acid sequences, polynucleotides, oligonucleotides, probes, primers, primer-specific portions, target-specific portions, tag sequences, and tag complement sequences.

In this application, a statement that one sequence is complementary to another sequence encompasses situations in which the two sequences have mismatches. Here, the term “sequence” encompasses, but is not limited to, nucleic acid sequences, polynucleotides, oligonucleotides, probes, primers, primer-specific portions, target-specific portions, tag sequences, and tag complement sequences. Despite the mismatches, the two sequences should selectively hybridize to one another under appropriate conditions.

The term “selectively hybridize” means that, for particular identical sequences, a substantial portion of the particular identical sequences hybridize to a given desired sequence or sequences, and a substantial portion of the particular identical sequences do not hybridize to other undesired sequences. A “substantial portion of the particular identical sequences” in each instance refers to a portion of the total number of the particular identical sequences, and it does not refer to a portion of an individual particular identical sequence. In certain embodiments, “a substantial portion of the particular identical sequences” means at least 90% of the particular identical sequences. In certain embodiments, “a substantial portion of the particular identical sequences” means at least 95% of the particular identical sequences.

In certain embodiments, the number of mismatches that may be present may vary in view of the complexity of the composition. Thus, in certain embodiments, fewer mismatches may be tolerated in a composition comprising DNA from an entire genome than a composition in which fewer DNA sequences are present. For example, in certain embodiments, with a given number of mismatches, a probe may more likely hybridize to undesired sequences in a composition with the entire genomic DNA than in a composition with fewer DNA sequences, when the same hybridization conditions are employed for both compositions. Thus, that given number of mismatches may be appropriate for the composition with fewer DNA sequences, but fewer mismatches may be more optimal for the composition with the entire genomic DNA.

In certain embodiments, sequences are complementary if they have no more than 20% mismatched nucleotides. In certain embodiments, sequences are complementary if they have no more than 15% mismatched nucleotides. In certain embodiments, sequences are complementary if they have no more than 10% mismatched nucleotides. In certain embodiments, sequences are complementary if they have no more than 5% mismatched nucleotides.

In this application, a statement that one sequence hybridizes or binds to another sequence encompasses situations where the entirety of both of the sequences hybridize or bind to one another, and situations where only a portion of one or both of the sequences hybridizes or binds to the entire other sequence or to a portion of the other sequence. Here, the term “sequence” encompasses, but is not limited to, nucleic acid sequences, polynucleotides, oligonucleotides, probes, primers, primer-specific portions, target-specific portions, tag sequences, and tag complement sequences.

The term label includes, but is not limited to, any moiety which can be attached to a nucleic acid and: (i) provides a detectable signal; (ii) interacts with a second label to modify the detectable signal provided by the second label, e.g. FRET (Fluorescent Resonance Energy Transfer); or (iii) provides a member of a binding complex or affinity set, e.g., affinity, antibody/antigen, ionic complexation, hapten/ligand, e.g. biotin/avidin.

“Mobility modifiers” of the present invention are any moieties that alter the migration of a polynucleotide in a mobility-dependent analysis technique, such as electrophoresis.

As used in this specification, the terms “mobility modifier cassette,” “mobility cassette,” and “cassette” refer to a polynucleotide attached to a mobility modifier that is capable of hybridizing to an analyte polynucleotide.

A “ligation agent” according to the present invention may comprise any number of enzymatic or chemical (i.e., non-enzymatic) agents that can effect ligation of nucleic acids to one another.

“Mobility-dependent analysis technique” refers to any analysis based on different rates of migration between different analytes. Exemplary mobility-dependent analyses include, but are not limited to, electrophoresis, mass spectroscopy, chromatography, sedimentation, gradient centrifugation, field-flow fractionation, and multi-stage extraction techniques.

In certain embodiments, the term “to a measurably lesser extent” encompasses situations in which the event in question is reduced at least 10 fold. In certain embodiments, the term “to a measurably lesser extent” encompasses situations in which the event in question is reduced at least 100 fold.

B. Certain Exemplary Components

In the following disclosure, the term “TNS/PNA” refers to either a target nucleic acid sequence or an analyte polynucleotide. In various embodiments, TNS/PNA's may include RNA and DNA. Exemplary RNA TNS/PNA's include, but are not limited to, mRNA, rRNA, tRNA, viral RNA, and variants of RNA, such as splicing variants. Exemplary DNA TNS/PNA's include, but are not limited to, genomic DNA, plasmid DNA, phage DNA, nucleolar DNA, mitochondrial DNA, and chloroplast DNA.

In certain embodiments, TNS/PNA's include, but are not limited to, cDNA, yeast artificial chromosomes (YAC's), bacterial artificial chromosomes (BAC's), other extrachromosomal DNA, and nucleic acid analogs. Exemplary nucleic acid analogs include, but are not limited to, LNAs, PNAs, PPG's, and other nucleic acid analogs.

In certain embodiments, TNS/PNA's include, but are not limited to, amplification products, ligation products, transcription products, reverse transcription products, primer extension products, methylated DNA, and cleavage products.

In certain embodiments, nucleic acids in a sample may be subjected to a cleavage procedure. Such cleavage procedures include, but are not limited to, restriction endonuclease digestion or the Flap Endonuclease (FEN) digestion of probes. For example, such FEN digestion is used as part of the Invader™ assay (Third Wave Technologies, Madison, Wis.). Such digestion employs a probe that is cleaved when a specific target is present. The presence of the specific nucleic acid sequence results in a cleavage product from the probe. In certain embodiments, such cleavage products may be targets or analytes.

In certain embodiments, two different analyte polynucleotides may differ by a single nucleotide, such as, e.g., two different single nucleotide polymorphisms. In certain embodiments, two different target nucleic acid sequences may differ by a single nucleotide, such as, e.g., two different single nucleotide polymorphisms.

A variety of methods are available for obtaining TNAS/PNA's for use with the compositions and methods of the present invention. When the TNAS/PNA is obtained through isolation from a biological matrix, certain isolation techniques include, but are not limited to, (1) organic extraction followed by ethanol precipitation, e.g., using a phenol/chloroform organic reagent (e.g., Ausubel et al., eds., Current Protocols in Molecular Biology Volume 1, Chapter 2, Section I, John Wiley & Sons, New York (1993)), in certain embodiments, using an automated DNA extractor, e.g., the Model 341 DNA Extractor available from Applied Biosystems (Foster City, Calif.); (2) stationary phase adsorption methods (e.g., Boom et al., U.S. Pat. No. 5,234,809; Walsh et al., Biotechniques 10(4): 506-513 (1991)); and (3) salt-induced DNA precipitation methods (e.g., Miller et al., Nucleic Acids Research, 16(3): 9-10 (1988)), such precipitation methods being typically referred to as “salting-out” methods. In certain embodiments, the above isolation methods may be preceded by an enzyme digestion step to help eliminate unwanted protein from the sample, e.g., digestion with proteinase K, or other like proteases. See, e.g., U.S. patent application Ser. No. 09/724,613.

In certain embodiments, a TNAS/PNA may be derived from any living, or once living, organism, including but not limited to prokaryote, eukaryote, plant, animal, and virus. In certain embodiments, the TNAS/PNA may originate from a nucleus of a cell, e.g., genomic DNA, or may be extranuclear nucleic acid, e.g., plasmid, mitrochondrial nucleic acid, various RNAs, and the like. In certain embodiments, if the sequence from the organism is RNA, it may be reverse-transcribed into a cDNA TNAS/PNA. Furthermore, in certain embodiments, the TNAS/PNA may be present in a double stranded or single stranded form.

Different TNAS/PNA's may be different portions of a single contiguous nucleic acid or may be on different nucleic acids. Different portions of a single contiguous nucleic acid may or may not overlap.

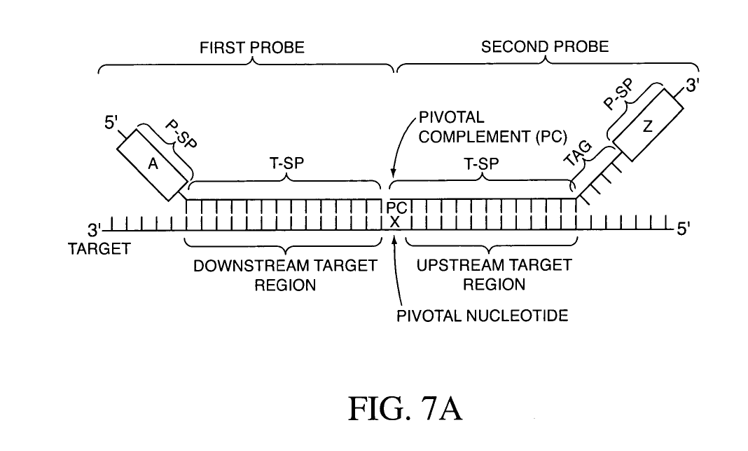

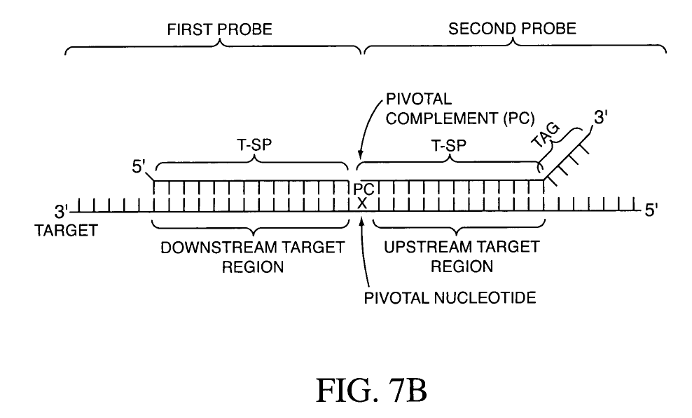

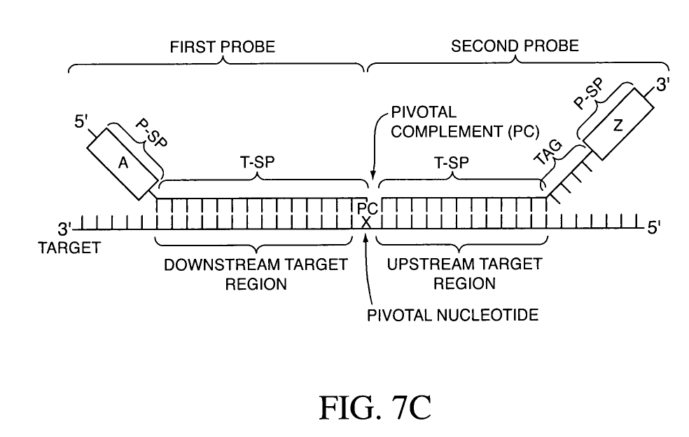

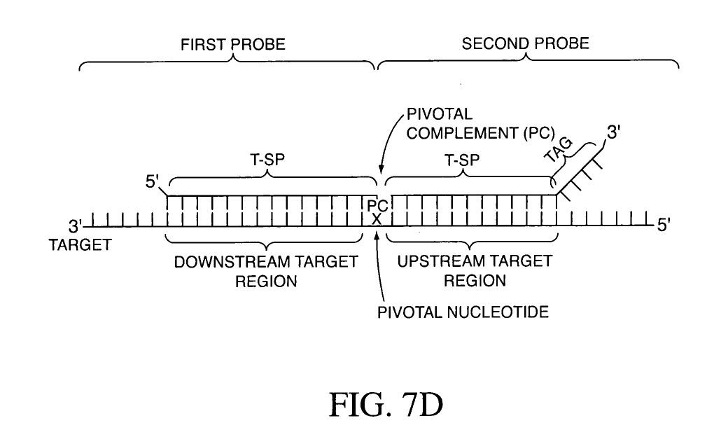

In certain embodiments, a TNAS/PNA comprises an upstream or 5′ region, a downstream or 3′ region, and a “pivotal nucleotide” located in the upstream region or the downstream region (see, e.g., FIGS. 7(A)-(D)). In certain embodiments, the pivotal nucleotide may be the nucleotide being detected by the probe set and may represent, for example, without limitation, a single polymorphic nucleotide in a multiallelic target locus. In certain embodiments, more than one pivotal nucleotide is present. In certain embodiments, one or more pivotal nucleotides is located in the upstream region, and one or more pivotal nucleotide is located in the downstream region. In certain embodiments, more than one pivotal nucleotides is located in the upstream region or the downstream region.

The person of ordinary skill will appreciate that while a TNAS/PNA is typically described as a single-stranded molecule, the opposing strand of a double-stranded molecule comprises a complementary sequence that may also be used as a target sequence.

A ligation probe set, according to certain embodiments, comprises two or more probes that comprise a target-specific portion (T-SP) that is designed to hybridize in a sequence-specific manner with a complementary region on a specific target nucleic acid sequence (see, e.g., first and second probes in FIGS. 7(A)-(D)). In certain embodiments, a probe of a ligation probe set may further comprise a primer-specific portion (P-SP or PSP), a tag sequence, all or part of a promoter or its complement, or a combination of these additional components. In certain embodiments, any of the probe's components may overlap any other probe component(s). For example, but without limitation, the target-specific portion may overlap, the primer-specific portion, the promoter or its complement, or both. Also, without limitation, the tag sequence may overlap with the target-specific portion or the primer specific-portion, or both.

In certain embodiments, at least one probe of a ligation probe set comprises the tag sequence located between the target-specific portion and the primer-specific portion (see, e.g., second probe in FIGS. 7(A) and (C)). In certain embodiments, the probe's tag sequence may comprise a sequence that is complementary to a portion of a mobility modifier cassette. In certain embodiments, the probe's tag sequence is not complementary with target sequences, primer sequences, or probe sequences.

The sequence-specific portions of probes are of sufficient length to permit specific annealing to complementary sequences in primers, mobility modifier cassettes, and targets as appropriate. In certain embodiments, the length of the tag sequences and target-specific portion are 6 to 35 nucleotides. Detailed descriptions of probe design that provide for sequence-specific annealing can be found, among other places, in Diffenbach and Dveksler, PCR Primer, A Laboratory Manual, Cold Spring Harbor Press, 1995, and Kwok et al., Nucl. Acid Res. 18:999-1005 (1990).

A ligation probe set according to certain embodiments comprises at least one first probe and at least one second probe that adjacently hybridize to the same target nucleic acid sequence. According to certain embodiments, a ligation probe set is designed so that the target-specific portion of the first probe will hybridize with the downstream target region (see, e.g., FIGS. 7(A)-(D)) and the target-specific portion of the second probe will hybridize with the upstream target region (see, e.g., FIGS. 7(A)-(D)). The sequence-specific portions of the probes are of sufficient length to permit specific annealing with complementary sequences in targets and primers, as appropriate. In certain embodiments, one of the at least one first probe and the at least one second probe in a probe set further comprises a tag sequence.

Under appropriate conditions, adjacently hybridized probes may be ligated together to form a ligation product, provided that they comprise appropriate reactive groups, for example, without limitation, a free 3′-hydroxyl or 5′-phosphate group.

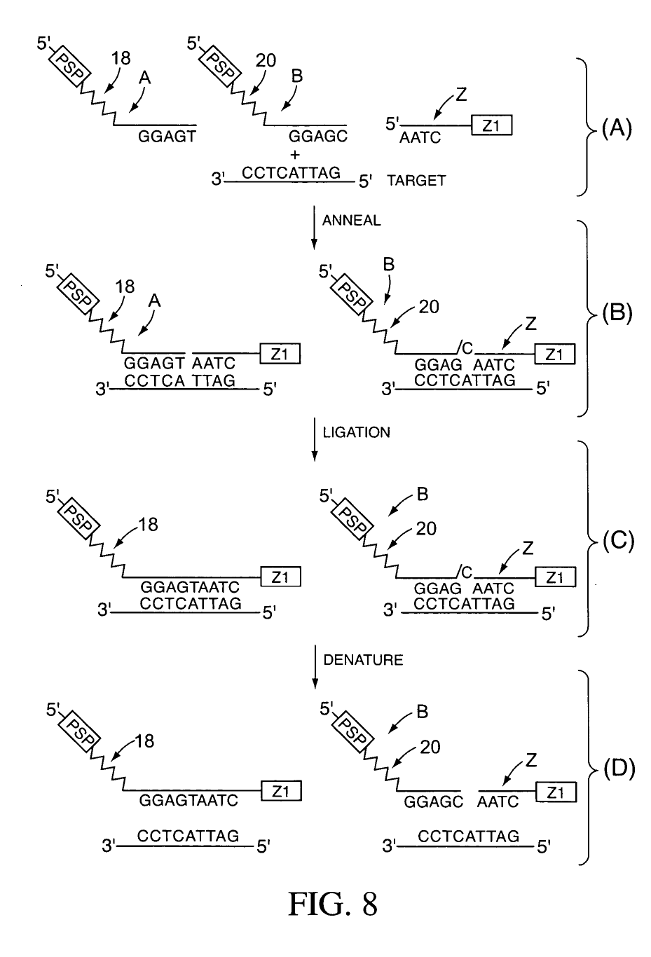

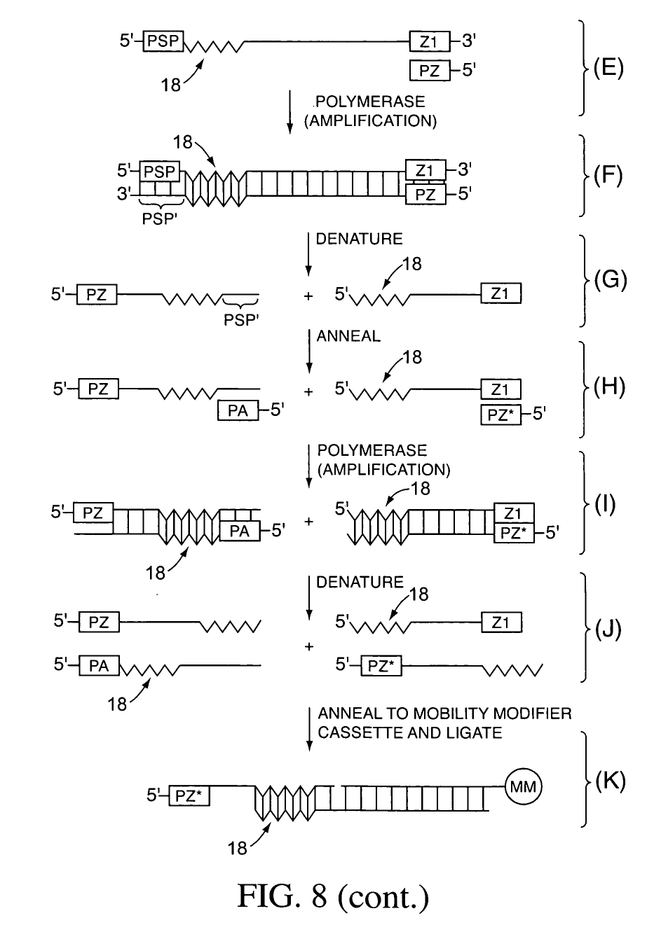

According to certain embodiments, some ligation probe sets may comprise more than one first probe or more than one second probe to allow sequence discrimination between target sequences that differ by one or more nucleotides (see, e.g., FIGS. 8-11).

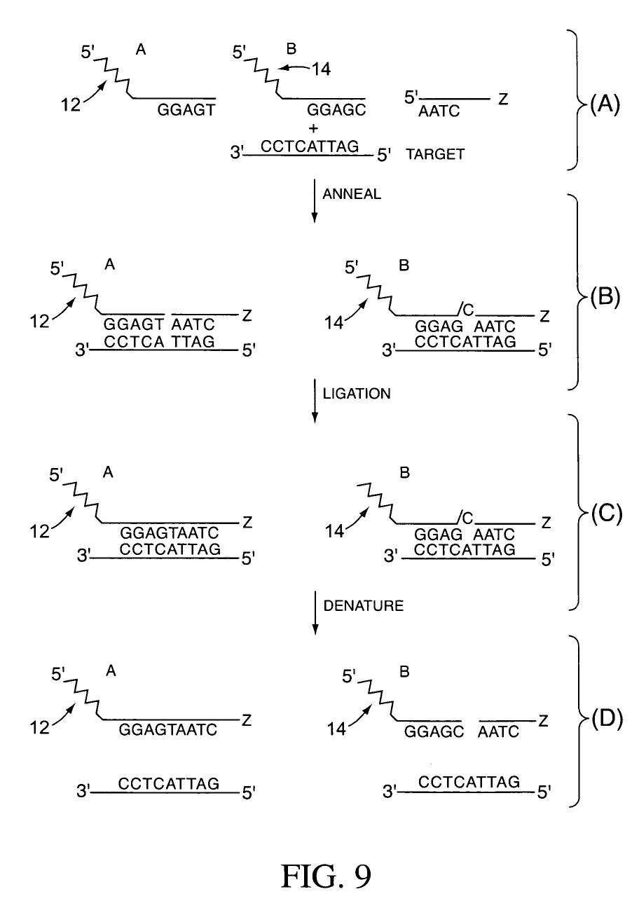

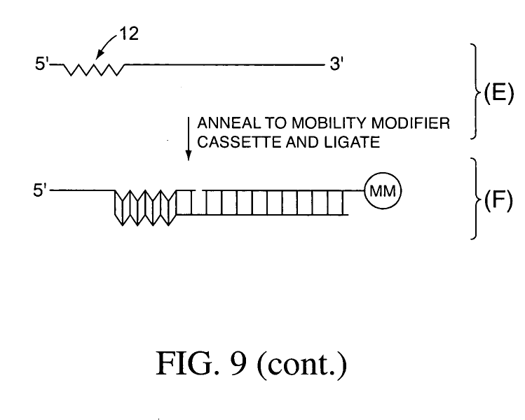

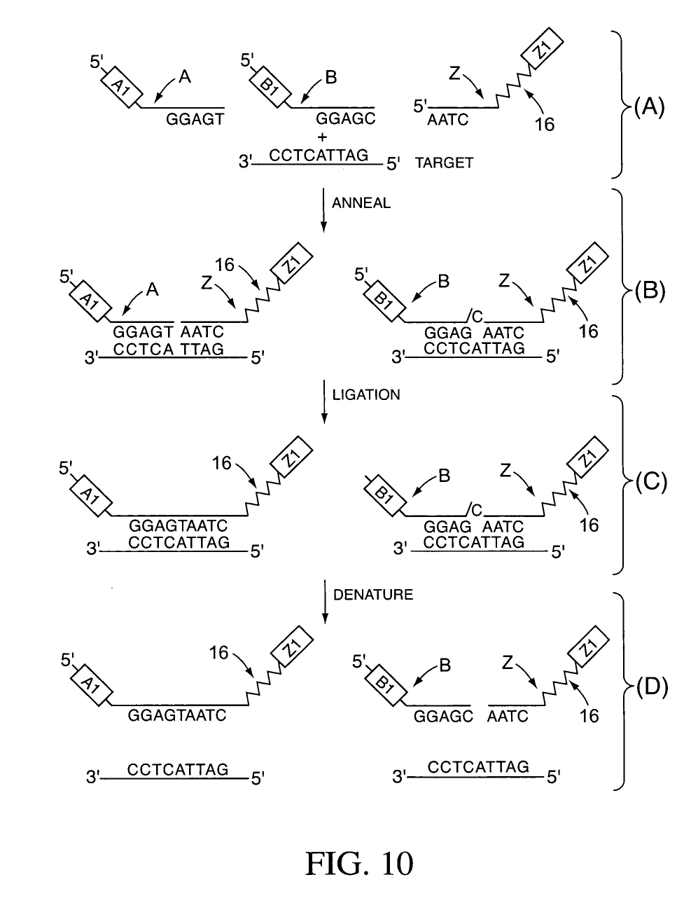

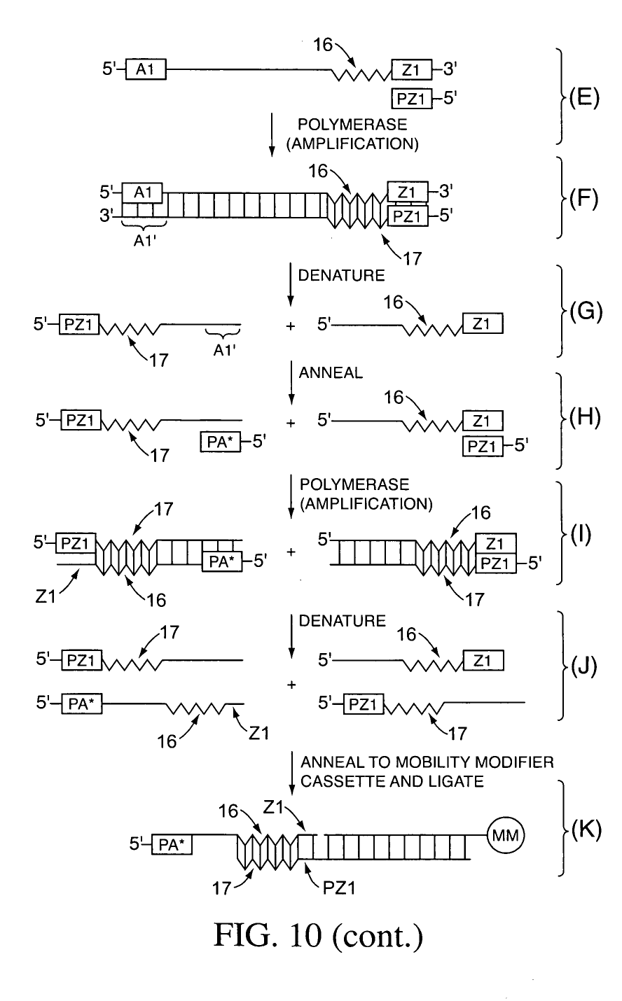

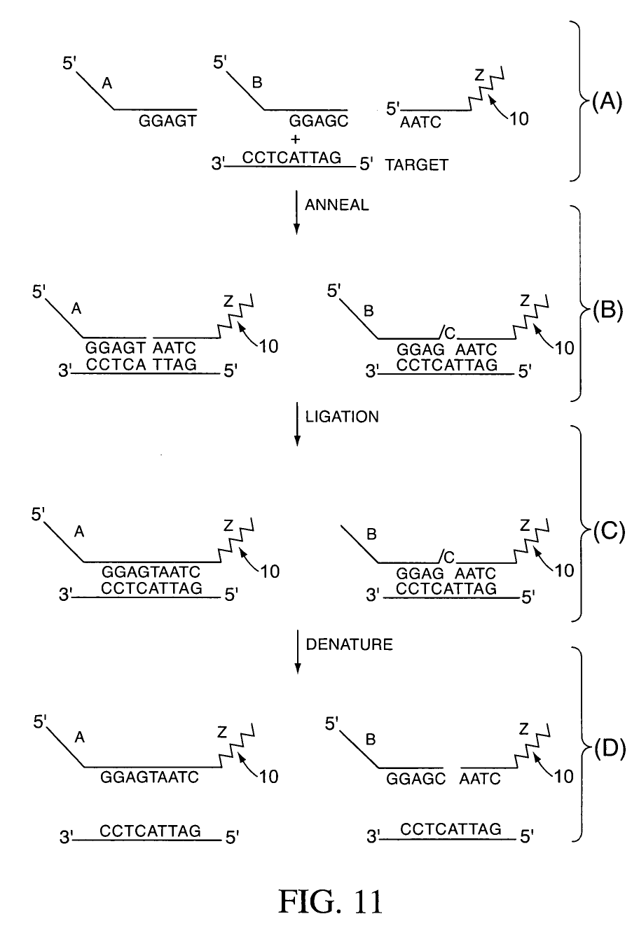

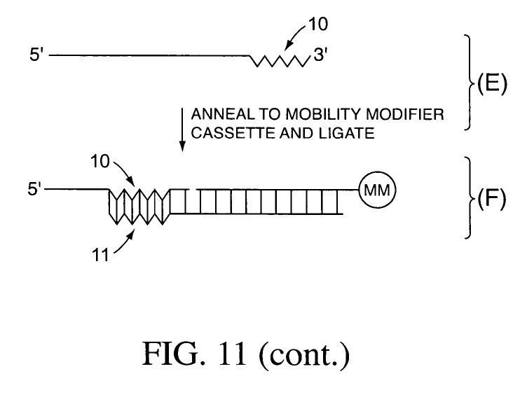

In certain embodiments, a nucleotide base complementary to the pivotal nucleotide (X), called the “pivotal complement” or “pivotal complement nucleotide,” is present on the proximal end of the second probe of the target-specific probe set (see, e.g., 5′ end (PC) of the second probe in FIGS. 7(A)-(B), and 3′ end (PC) of the first probe in FIGS. 7(C)-(D)). In certain embodiments, the second probe further comprises a tag sequence (see, e.g., FIGS. 7(A)-(D)). In certain embodiments, the second probe does not comprise a tag sequence and the first probe comprises a tag sequence. In certain embodiments, the first probe may comprise a pivotal complement and a tag sequence (see, e.g., FIG. 8, probes A and B comprising tag sequences 18 and 20, respectively; and FIG. 9, probes A and B comprising tag sequences 12 and 14, respectively). In certain embodiments, the first probe may comprise a pivotal complement and the second probe may comprise a tag sequence (see, e.g., FIGS. 10 and 11; both FIGS. 10 and 11 show first probes A and B with pivotal complements; FIG. 10 shows second probe Z comprising tag sequence 16; and FIG. 11 shows second probe Z comprising tag sequence 10).

The skilled artisan will appreciate that, in various embodiments, the pivotal nucleotide(s) may be located anywhere in the target sequence and that likewise, the pivotal complement may be located anywhere within the target-specific portion of the probe(s). For example, according to various embodiments, the pivotal complement may be located at the 3′ end of a probe, at the 5′ end of a probe, or anywhere between the 3′ end and the 5′ end of a probe.

In certain embodiments, when the first and second probes of the ligation probe set are hybridized to the appropriate upstream and downstream target regions, and when the pivotal complement is at the 5′ end of one probe or the 3′ end of the other probe, and the pivotal complement is base-paired with the pivotal nucleotide on the target sequence, the hybridized first and second probes may be ligated together to form a ligation product (see, e.g., FIGS. 8(B)-(C), 9(B)-(C), 10(B)-(C), and 11(B)-(C)). In the example shown in FIGS. 8(B)-(C) 9(B)-(C), 10(B)-(C), and 11(B)-(C), a mismatched base at the pivotal nucleotide, however, interferes with ligation, even if both probes are otherwise fully hybridized to their respective target regions.

In certain embodiments, other mechanisms may be employed to avoid ligation of probes that do not include the correct complementary nucleotide at the pivotal complement. For example, in certain embodiments, conditions may be employed such that a probe of a ligation probe set will hybridize to the target sequence to a measurably lesser extent if there is a mismatch at the pivotal nucleotide. Thus, in such embodiments, such non-hybridized probes will not be ligated to the other probe in the probe set.

In certain embodiments, the first probes and second probes in a ligation probe set are designed with similar melting temperatures (Tₘ). Where a probe includes a pivotal complement, in certain embodiments, the Tₘ for the probe(s) comprising the pivotal complement(s) of the target pivotal nucleotide sought will be approximately 4° C. to 15° C. lower than the other probe(s) that do not contain the pivotal complement in the probe set. In certain such embodiments, the probe comprising the pivotal complement(s) will also be designed with a Tₘ near the ligation temperature. Thus, a probe with a mismatched nucleotide will more readily dissociate from the target at the ligation temperature. The ligation temperature, therefore, in certain embodiments provides another way to discriminate between, for example, multiple potential alleles in the target.

Further, in certain embodiments, ligation probe sets do not comprise a pivotal complement at the terminus of the first or the second probe (e.g., at the 3′ end or the 5′ end of the first or second probe). Rather, the pivotal complement is located somewhere between the 5′ end and the 3′ end of the first or second probe. In certain such embodiments, probes with target-specific portions that are fully complementary with their respective target regions will hybridize under high stringency conditions. Probes with one or more mismatched bases in the target-specific portion, by contrast, will hybridize to their respective target region to a measurably lesser extent. Both the first probe and the second probe must be hybridized to the target for a ligation product to be generated.

In certain embodiments, highly related sequences that differ by as little as a single nucleotide can be distinguished. For example, according to certain embodiments, one can distinguish the two potential alleles in a biallelic locus as follows. One can combine a ligation probe set comprising two first probes differing in their pivotal complements (see, e.g., probes A and B in FIGS. 8(A), 9(A), 10(A), and 11(A)) and one second probe (see, e.g., probe Z in FIGS. 8(A), 9(A), 10(A), and 11(A)) with the sample containing the target. In certain embodiments, the two first probes comprise different tags (see, e.g., probes A and B in FIG. 8(A), comprising tags 18 and 20, respectively, and probes A and B in FIG. 9(A), comprising tags 12 and 14, respectively). All probes of the ligation probe set will hybridize with the target sequence under appropriate conditions (see, e.g., FIGS. 8(B), 9(B), 10(B), and 11(B). Only the first probe with the hybridized pivotal complement, however, will be ligated with the hybridized second probe (see, e.g., FIGS. 8(C), 9(C), 10(C), and 11(C)). Thus, if only one allele is present in the sample, only one ligation product for that target will be generated (see, e.g., ligation product of probes A and Z in FIGS. 8(D), 9(D), 10(D), and 11(D)). Both ligation products would be formed in a sample from a heterozygous individual. In certain embodiments, ligation of probes with a pivotal complement that is not complementary to the pivotal nucleotide may occur, but such ligation occurs to a measurably lesser extent than ligation of probes with a pivotal complement that is complementary to the pivotal nucleotide.

In certain embodiments, one of the first probe or the second probe may contain a pivotal complement and the other of the first probe or the second probe may contain a tag sequence. See, e.g., FIGS. 10 and 11.

In certain embodiments, one of the first or second probes of a ligation probe set may include a tag sequence and the first and second probes may not include primer-specific portions. In certain such embodiments, at least one first probe of a probe set comprises a pivotal complement and a tag sequence and at least one second probe of a probe set comprises a label (see, e.g., FIG. 9). In other such embodiments, at least one first probe of a probe set comprises a pivotal complement and a label and at least one second probe of a probe set comprises a tag sequence (see, e.g., FIG. 11).

A primer set according to certain embodiments comprises at least one primer capable of hybridizing with the primer-specific portion of at least one probe of a ligation probe set. In certain embodiments, a primer set comprises at least one first primer and at least one second primer, wherein the at least one first primer specifically hybridizes with one probe of a ligation probe set (or a complement of such a probe) and the at least one second primer of the primer set specifically hybridizes with a second probe of the same ligation probe set (or a complement of such a probe). In certain embodiments, at least one primer of a primer set further comprises all or part of a promoter sequence or its complement. In certain embodiments, the first and second primers of a primer set have different hybridization temperatures, to permit temperature-based asymmetric PCR reactions.

The skilled artisan will appreciate that while the probes and primers of the invention may be described in the singular form, a plurality of probes or primers may be encompassed by the singular term, as will be apparent from the context. Thus, for example, in certain embodiments, a ligation probe set typically comprises a plurality of first probes and a plurality of second probes.

The criteria for designing sequence-specific primers and probes are well known to persons of ordinary skill in the art. Detailed descriptions of primer design that provide for sequence-specific annealing can be found, among other places, in Diffenbach and Dveksler, PCR Primer, A Laboratory Manual, Cold Spring Harbor Press, 1995, and Kwok et al. (Nucl. Acid Res. 18:999-1005, 1990). The sequence-specific portions of the primers are of sufficient length to permit specific annealing to complementary sequences in ligation products and amplification products, as appropriate.

In embodiments that employ a promoter sequence, the promoter sequence or its complement will be of sufficient length to permit an appropriate polymerase to interact with it. Detailed descriptions of sequences that are sufficiently long for polymerase interaction can be found in, among other places, Sambrook and Russell.

According to certain embodiments, a primer set of the present invention comprises at least one second primer. In certain embodiments, the second primer in that primer set is designed to hybridize with a 3′ primer-specific portion of a ligation or amplification product in a sequence-specific manner. In certain embodiments, the primer set further comprises at least one first primer. In certain embodiments, the first primer of a primer set is designed to hybridize with the complement of the 5′ primer-specific portion of that same ligation or amplification product in a sequence-specific manner. In certain embodiments, at least one primer of the primer set comprises a promoter sequence or its complement or a portion of a promoter sequence or its complement. For a discussion of primers comprising promoter sequences, see, e.g., Sambrook and Russell. In certain embodiments, at least one primer of the primer set further comprises a label. In certain embodiments, labels are fluorescent dyes attached to a nucleotide(s) in the primer (see, e.g., L. Kricka, Nonisotopic DNA Probe Techniques, Academic Press, San Diego, Calif. (1992)). In certain embodiments, a label is attached to the primer in such a way as to not to interfere with sequence-specific hybridization or amplification.

A universal primer or primer set may be employed according to certain embodiments. In certain embodiments, a universal primer or a universal primer set hybridizes with two or more of the probes, ligation products, or amplification products in a reaction, as appropriate. When universal primer sets are used in certain amplification reactions, such as, but not limited to, PCR, qualitative or quantitative results may be obtained for a broad range of template concentrations.

Use of labels can be accomplished using any one of a large number of known techniques employing known labels, linkages, linking groups, reagents, reaction conditions, and analysis and purification methods. Labels include, but are not limited to, light-emitting or light-absorbing compounds which generate or quench a detectable fluorescent, chemiluminescent, or bioluminescent signal (see, e.g., Kricka, L. in Nonisotopic DNA Probe Techniques (1992), Academic Press, San Diego, pp. 3-28). Fluorescent reporter dyes useful as labels include, but are not limited to, fluoresceins (see, e.g., U.S. Pat. Nos. 5,188,934; 6,008,379; and 6,020,481), rhodamines (see, e.g., U.S. Pat. Nos. 5,366,860; 5,847,162; 5,936,087; 6,051,719; and 6,191,278), benzophenoxazines (see, e.g., U.S. Pat. No. 6,140,500), energy-transfer fluorescent dyes, comprising pairs of donors and acceptors (see, e.g., U.S. Pat. Nos. 5,863,727; 5,800,996; and 5,945,526), and cyanines (see, e.g., Kubista, WO 97/45539), as well as any other fluorescent moiety capable of generating a detectable signal. Examples of fluorescein dyes include, but are not limited to, 6-carboxyfluorescein; 2′,4′,1,4,-tetrachlorofluorescein; and 2′,4′,5′,7′,1,4-hexachlorofluorescein.

Labels also include, but are not limited to, quantum dots. “Quantum dots” refer to semiconductor nanocrystalline compounds capable of emitting a second energy in response to exposure to a first energy. Typically, the energy emitted by a single quantum dot always has the same predictable wavelength. Exemplary semiconductor nanocrystalline compounds include, but are not limited to, crystals of CdSe, CdS, and ZnS. Suitable quantum dots according to certain embodiments are described, e.g., in U.S. Pat. Nos. 5,990,479 and 6,207,392 B1, and in “Quantum-dot-tagged microbeads for multiplexed optical coding of biomolecules,” Han et al., Nature Biotechnology, 19:631-635 (2001).

Labels also include, but are not limited to, phosphors and luminescent molecules. The term “reporter group” as used herein refers to any tag, label, or identifiable moiety. The skilled artisan will appreciate that many reporter groups may be used. For example, reporter groups include, but are not limited to, fluorophores, radioisotopes, chromogens, enzymes, antigens, heavy metals, dyes, magnetic probes, phosphorescence groups, chemiluminescent groups, and electrochemical detection moieties. Exemplary fluorophores that are used as reporter groups include, but are not limited to, rhodamine, cyanine 3 (Cy 3), cyanine 5 (Cy 5), fluorescein, ViC™, Liz™, Tamra™, 5-FaM™, 6-Fam™, and Texas Red (Molecular Probes). (ViC™, LiZ™, Tamra™, 5-Fam™, and 6-Fam™ are all available from Applied Biosystems, Foster City, Calif.) Exemplary radioisotopes include, but are not limited to, ³²P, ³³P, and ³⁵S. Labels also include elements of multi-element indirect reporter systems, e.g., biotin/avidin, antibody/antigen, ligand/receptor, enzyme/substrate, and the like, in which the element interacts with other elements of the system in order to effect a detectable signal. One exemplary multi-element reporter system includes a biotin reporter group attached to a primer and an avidin conjugated with a fluorescent label.

The skilled artisan will appreciate that, in certain embodiments, one or more of the primers, probes, deoxyribonucleotide triphosphates, ribonucleotide triphosphates disclosed herein may further comprise one or more labels. Detailed protocols for methods of attaching labels to oligonucleotides and polynucleotides can be found in, among other places, G. T. Hermanson, Bioconjugate Techniques, Academic Press, San Diego, Calif. (1996) and S. L. Beaucage et al., Current Protocols in Nucleic Acid Chemistry, John Wiley & Sons, New York, N.Y. (2000).

Certain non-radioactive labelling methods, techniques, and reagents are reviewed in: Non-Radioactive Labelling, A Practical Introduction, Garman, A. J. (1997) Academic Press, San Diego.

In certain embodiments, mobility modifiers may be nucleotides of different lengths effecting different mobilities. In certain embodiments, mobility modifiers may be non-nucleotide polymers, such as a polyethylene oxide (PEO), polyglycolic acid, polyurethane polymers, polypeptides, or oligosaccharides, as non-limiting examples. In certain embodiments, mobility modifiers may work by adding size to a polynucleotide, or by increasing the “drag” of the molecule during migration through a medium without substantially adding to the size. Certain mobility modifiers such as PEO's have been described, e.g., in U.S. Pat. Nos. 5,470,705; 5,580,732; 5,624,800; and 5,989,871.

In certain embodiments, a mobility cassette comprises a mobility modifier attached to a first strand of a double-stranded polynucleotide. The second strand of the double-stranded polynucleotide comprises a tag complement sequence. The tag complement sequence is designed to be complementary to tag sequence of an analyte polynucleotide such that, when the tag complement sequence is hybridized to the tag sequence of an analyte polynucleotide, the first strand of the double-stranded polynucleotide is suitable for ligation to the analyte polynucleotide. When the first strand is ligated to the analyte polynucleotide, a single contiguous polynucleotide attached to a mobility modifier is formed.

Linkage of polymers such as PEO's to polynucleotides is well known in the art. Standard DNA chemistry linkages are described, e.g., in Grossman et al., Nucleic Acids Research, 22(21):4527-34 (1994).

Certain embodiments include a ligation agent. For example, ligase is an enzymatic ligation agent that, under appropriate conditions, forms phosphodiester bonds between the 3′-OH and the 5′-phosphate of adjacent nucleotides in DNA or RNA molecules, or hybrids. Exemplary ligases include, but are not limited to, Tth K294R ligase and Tsp AK16D ligase. See, e.g., Luo et al., Nucleic Acids Res., 24(14):3071-3078 (1996); Tong et al., Nucleic Acids Res., 27(3):788-794 (1999); and Published PCT Application No. WO 00/26381. Temperature sensitive ligases, include, but are not limited to, T4 DNA ligase T7 DNA ligase, and E. coli ligase. In certain embodiments, thermostable ligases include, but are not limited to, Taq ligase, Tth ligase, Tsc ligase, and Pfu ligase. Certain thermostable ligases may be obtained from thermophilic or hyperthermophilic organisms, including but not limited to, prokaryotic, eucaryotic, or archael organisms. Certain RNA ligases may be employed in certain embodiments. Exemplary, but nonlimiting examples, of RNA ligases include, but are not limited to T4 RNA ligase and T. brucei RNA ligase. In certain embodiments, the ligase is a RNA dependent DNA ligase, which may be employed with RNA template and DNA ligation probes. An exemplary, but nonlimiting example, of a ligase with such RNA dependent DNA ligase activity is T4 DNA ligase. In certain embodiments, the ligation agent is an “activating” or reducing agent.

Chemical ligation agents include, without limitation, activating, condensing, and reducing agents, such as carbodiimide, cyanogen bromide (BrCN), N-cyanoimidazole, imidazole, 1-methylimidazole/carbodiimide/cystamine, dithiothreitol (DTT) and ultraviolet light. Autoligation, i.e., spontaneous ligation in the absence of a ligating agent, is also within the scope of certain embodiments of the invention. Detailed protocols for chemical ligation methods and descriptions of appropriate reactive groups can be found, among other places, in Xu et al., Nucleic Acid Res., 27:875-81 (1999); Gryaznov and Letsinger, Nucleic Acid Res. 21:1403-08 (1993); Gryaznov et al., Nucleic Acid Res. 22:2366-69 (1994); Kanaya and Yanagawa, Biochemistry 25:7423-30 (1986); Luebke and Dervan, Nucleic Acids Res. 20:3005-09 (1992); Sievers and von Kiedrowski, Nature 369:221-24 (1994); Liu and Taylor, Nucleic Acids Res. 26:3300-04 (1999); Wang and Kool, Nucleic Acids Res. 22:2326-33 (1994); Purmal et al., Nucleic Acids Res. 20:3713-19 (1992); Ashley and Kushlan, Biochemistry 30:2927-33 (1991); Chu and Orgel, Nucleic Acids Res. 16:3671-91 (1988); Sokolova et al., FEBS Letters 232:153-55 (1988); Naylor and Gilham, Biochemistry 5:2722-28 (1966); and U.S. Pat. No. 5,476,930.

In certain embodiments, at least one polymerase is included. In certain embodiments, at least one thermostable polymerase is included. Exemplary thermostable polymerases, include, but are not limited to, Taq polymerase, Pfx polymerase, Pfu polymerase, Vent® polymerase, Deep Vent™ polymerase, Pwo polymerase, Tth polymerase, UITma polymerase and enzymatically active mutants and variants thereof. Descriptions of these polymerases may be found, among other places, at the world wide web URL: the-scientist.com/yr1998/jan/profile 1_{—}980105. html; at the world wide web URL: the-scientist.com/yr2001/jan/profile_{—}010903. html; at the world wide web URL: the-scientist.com/yr2001/sep/profile2_{—}010903. html; at the article The Scientist 12(1):17 (Jan. 5, 1998); and at the article The Scientist 15(17):1 (Sep. 3, 2001).

The skilled artisan will appreciate that the complement of the disclosed probe, target, analyte, and primer sequences, or combinations thereof, may be employed in certain embodiments of the invention. For example, without limitation, a genomic DNA sample may comprise both the target sequence and its complement. Thus, in certain embodiments, when a genomic sample is denatured, both the target sequence and its complement are present in the sample as single-stranded sequences. In certain embodiments, the probes may be designed to specifically hybridize to an appropriate sequence, either the target or its complement.

C. Certain Exemplary Component Methods

Ligation according to the present invention comprises any enzymatic or chemical process wherein an internucleotide linkage is formed between the opposing ends of nucleic acid sequences that are adjacently hybridized to a template. Additionally, the opposing ends of the annealed nucleic acid sequences should be suitable for ligation (suitability for ligation is a function of the ligation method employed). The internucleotide linkage may include, but is not limited to, phosphodiester bond formation. Such bond formation may include, without limitation, those created enzymatically by a DNA or RNA ligase, such as bacteriophage T4 DNA ligase, T4 RNA ligase, T7 DNA ligase, Thermus thermophilus (Tth) ligase, Thermus aquaticus (Taq) ligase, or Pyrococcus furiosus (Pfu) ligase. Other internucleotide linkages include, without limitation, covalent bond formation between appropriate reactive groups such as between an α-haloacyl group and a phosphothioate group to form a thiophosphorylacetylamino group; and between a phosphorothioate, a tosylate, or iodide group to form a 5′-phosphorothioester or pyrophosphate linkages.

In certain embodiments, chemical ligation may, under appropriate conditions, occur spontaneously such as by autoligation. Alternatively, in certain embodiments, “activating” or reducing agents may be used. Examples of activating agents and reducing agents include, without limitation, carbodiimide, cyanogen bromide (BrCN), imidazole, 1-methylimidazole/carbodiimide/cystamine, N-cyanoimidazole, dithiothreitol (DTT) and ultraviolet light. Nonenzymatic ligation according to certain embodiments may utilize specific reactive groups on the respective 3′ and 5′ ends of the aligned probes.

In certain embodiments, ligation generally comprises at least one cycle of ligation, for example, the sequential procedures of: hybridizing the target-specific portions of a first probe and a second probe, that are suitable for ligation, to their respective complementary regions on a target nucleic acid sequence; ligating the 3′ end of the first probe with the 5′ end of the second probe to form a ligation product; and denaturing the nucleic acid duplex to separate the ligation product from the target nucleic acid sequence. The cycle may or may not be repeated. For example, without limitation, by thermocycling the ligation reaction to linearly increase the amount of ligation product.

According to certain embodiments, one may use ligation techniques such as gap-filling ligation, including, without limitation, gap-filling OLA and LCR, bridging oligonucleotide ligation, FEN-LCR, and correction ligation. Descriptions of these techniques can be found, among other places, in U.S. Pat. No. 5,185,243, published European Patent Applications EP 320308 and EP 439182, published PCT Patent Applications WO 90/01069 and WO 02/02823, and U.S. patent application Ser. No. 09/898,323 (U.S. Pat. No. 6,511,810).

In certain embodiments, after ligation, the composition may be used directly in a subsequent amplification reaction. In certain embodiments, after ligation, the composition may be subjected to a purification technique that results in a composition that includes less than all of the components that may have been present after the at least one cycle of ligation. For example, in certain embodiments, one may purify the ligation product.

Purifying the ligation product according to certain embodiments comprises any process that removes at least some unligated probes, target nucleic acid sequences, enzymes, and/or accessory agents from the ligation reaction composition following at least one cycle of ligation. Such processes include, but are not limited to, molecular weight/size exclusion processes, e.g., gel filtration chromatography or dialysis, sequence-specific hybridization-based pullout methods, affinity capture techniques, precipitation, adsorption, or other nucleic acid purification techniques.

In certain embodiments that employ a subsequent amplification after ligation, the skilled artisan will appreciate that purifying the ligation product prior to amplification in certain embodiments reduces the quantity of primers needed to amplify the ligation product, thus reducing the cost of detecting a target sequence. Also, in certain embodiments, purifying the ligation product prior to amplification may decrease possible side reactions during amplification and may reduce competition from unligated probes during hybridization. Certain exemplary purification techniques are discussed, for example, in U.S. Patent Application No. 60/427,818, filed Nov. 19, 2002, and U.S. Patent Application No. 60/445,636, filed Feb. 7, 2003.

Hybridization-based pullout (HBP) according to certain embodiments of the present invention comprises a process wherein a nucleotide sequence complementary to at least a portion of one probe (or its complement), for example, the primer-specific portion, is bound or immobilized to a solid or particulate pullout support (see, e.g., U.S. Pat. No. 6,124,092 and PCT Publication No. WO 02/10373, published Feb. 7, 2003). In certain embodiments, a composition comprising a ligation product, target sequences, and unligated probes is exposed to the pullout support. The ligation product, under appropriate conditions, hybridizes, with the support-bound sequences. The unbound components of the composition are removed, purifying the ligation products from those composition components that do not contain sequences complementary to the sequence on the pullout support. In certain embodiments involving amplification, one subsequently removes the purified ligation products from the support and combines them with at least one primer set to form a first amplification reaction composition. The skilled artisan will appreciate that, in certain embodiments, additional cycles of HBP using different complementary sequences on the pullout support may remove all or substantially all of the unligated probes, further purifying the ligation product.

Amplification according to the present invention encompasses a broad range of techniques for amplifying nucleic acid sequences, either linearly or exponentially. Exemplary amplification techniques include, but are not limited to, PCR or any other method employing a primer extension step, and transcription or any other method of generating at least one RNA transcription product. Other nonlimiting examples of amplification are ligase detection reaction (LDR), and ligase chain reaction (LCR). Amplification methods may comprise thermal-cycling or may be performed isothermally. The term “amplification product” includes products from any number of cycles of amplification reactions, primer extension reactions, and RNA transcription reactions, unless otherwise apparent from the context.

In certain embodiments, amplification methods comprise at least one cycle of amplification, for example, but not limited to, the sequential procedures of: hybridizing primers to primer-specific portions of the ligation product or amplification products from any number of cycles of an amplification reaction; synthesizing a strand of nucleotides in a template-dependent manner using a polymerase; and denaturing the newly-formed nucleic acid duplex to separate the strands. The cycle may or may not be repeated. In certain embodiments, amplification methods comprise at least one cycle of amplification, for example, but not limited to, the sequential procedures of: interaction of a polymerase with a promoter; synthesizing a strand of nucleotides in a template-dependent manner using a polymerase; and denaturing the newly-formed nucleic acid duplex to separate the strands. The cycle may or may not be repeated.

Descriptions of certain amplification techniques can be found, among other places, in H. Ehrlich et al., Science, 252:1643-50 (1991), M. Innis et al., PCR Protocols: A Guide to Methods and Applications, Academic Press, New York, N.Y. (1990), R. Favis et al., Nature Biotechnology 18:561-64 (2000), and H. F. Rabenau et al., Infection 28:97-102 (2000); Sambrook et al., Molecular Cloning: A Laboratory Manual, Cold Spring Harbor Press, Cold Spring Harbor, N.Y. (2000); Ausbel et al., Current Protocols in Molecular Biology (1993), including supplements through April 2001, John Wiley & Sons, Somerset, N.J.

Primer extension according to the present invention comprises an amplification process comprising elongating a primer that is annealed to a template in the 5′ to 3′ direction using a template-dependent polymerase. According to certain embodiments, with appropriate buffers, salts, pH, temperature, and nucleotide triphosphates, including analogs and derivatives thereof, a template dependent polymerase incorporates nucleotides complementary to the template strand starting at the 3′-end of an annealed primer, to generate a complementary strand. Detailed descriptions of primer extension according to certain embodiments can be found, among other places in Sambrook et al., Sambrook and Russell, and Ausbel et al.

Transcription according to certain embodiments comprises an amplification process comprising an RNA polymerase interacting with a promoter on a single- or double-stranded template and generating a RNA polymer in a 5′ to 3′ direction. In certain embodiments, the transcription reaction composition further comprises transcription factors. RNA polymerases, including but not limited to T3, T7, and SP6 polymerases, according to certain embodiments, can interact with either single-stranded or double-stranded promoters. Detailed descriptions of transcription according to certain embodiments can be found, among other places in Sambrook et al., Sambrook and Russell, and Ausbel et al.

Certain embodiments of amplification may employ multiplex PCR, in which multiple target sequences are simultaneously amplified (see, e.g., H. Geada et al., Forensic Sci. Int. 108:31-37 (2000) and D. G. Wang et al., Science 280:1077-82 (1998)).

In certain embodiments, one employs asymmetric PCR. According to certain embodiments, asymmetric PCR comprises an amplification reaction composition comprising (i) at least one primer set in which there is an excess of one primer (relative to the other primer in the primer set); (ii) at least one primer set that comprises only a first primer or only a second primer; (iii) at least one primer set that, during given amplification conditions, comprises a primer that results in amplification of one strand and comprises another primer that is disabled; or (iv) at least one primer set that meets the description of both (i) and (iii) above. Consequently, when the ligation product is amplified, an excess of one strand of the amplification product (relative to its complement) is generated.

In certain embodiments, one may use at least one primer set wherein the the melting temperature (Tm₅₀) of one of the primers is higher than the Tm₅₀ of the other primer. Such embodiments have been called asynchronous PCR (A-PCR). See, e.g., U.S. patent application Ser. No. 09/875,211, filed Jun. 5, 2001 (U.S. Patent Application Publication No. 2003/0207266 A1). In certain embodiments, the Tm₅₀ of the first primer is at least 4-15° C. different from the Tm₅₀ of the second primer. In certain embodiments, the Tm₅₀ of the first primer is at least 8-15° C. different from the Tm₅₀ of the second primer. In certain embodiments, the Tm₅₀ of the first primer is at least 10-15° C. different from the Tm₅₀ of the second primer. In certain embodiments, the Tm₅₀ of the first primer is at least 10-12° C. different from the Tm₅₀ of the second primer. In certain embodiments, in at least one primer set, the melting temperature Tm₅₀ of the at least one first primer differs from the melting temperature of the at least one second primer by at least about 4° C., by at least about 8° C., by at least about 10° C., or by at least about 12° C.

In certain embodiments of A-PCR, in addition to the difference in Tm50 of the primers in a primer set, there is also an excess of one primer relative to the other primer in the primer set. In certain embodiments, there is a five to twenty-fold excess of one primer relative to the other primer in the primer set. In certain embodiments of A-PCR, the primer concentration is at least 50 mM.

In A-PCR according to certain embodiments, one may use conventional PCR in the first cycles such that both primers anneal and both strands are amplified. By raising the temperature in subsequent cycles, however, one may disable the primer with the lower Tm such that only one strand is amplified. Thus, the subsequent cycles of A-PCR in which the primer with the lower Tm is disabled result in asymmetric amplification. Consequently, when the ligation product is amplified, an excess of one strand of the amplification product (relative to its complement) is generated.

According to certain embodiments of A-PCR, the level of amplification can be controlled by changing the number of cycles during the first phase of conventional PCR cycling. In such embodiments, by changing the number of initial conventional cycles, one may vary the amount of the double strands that are subjected to the subsequent cycles of PCR at the higher temperature in which the primer with the lower Tm is disabled.

In certain embodiments, an A-PCR protocol may comprise use of a pair of primers, each of which has a concentration of at least 50 mM. In certain embodiments, conventional PCR, in which both primers result in amplification, is performed for the first 20-30 cycles. In certain embodiments, after 20-30 cycles of conventional PCR, the annealing temperature increases to 66-70° C., and PCR is performed for 5 to 40 cycles at the higher annealing temperature. In such embodiments, the lower Tm primer is disabled during such 5 to 40 cycles at higher annealing temperature. In such embodiments, asymmetric amplification occurs during the second phase of PCR cycles at a higher annealing temperature.

In certain embodiments, one employs asymmetric reamplification. According to certain embodiments, asymmetric reamplification comprises generating single-stranded amplification product in a second amplification process. In certain embodiments, the double-stranded amplification product of a first amplification process serves as the amplification target in the asymmetric reamplification process. In certain embodiments, one may achieve asymmetric reamplification using asynchronous PCR in which initial cycles of PCR conventionally amplify two strands and subsequent cycles are performed at a higher annealing temperature that disables one of the primers of a primer set as discussed above. In certain embodiments, the second amplification reaction composition comprises at least one primer set which comprises the at least one first primer, or the at least one second primer of a primer set, but typically not both. The skilled artisan understands that, in certain embodiments, asymmetric reamplification will also eventually occur if the primers in the primer set are not present in an equimolar ratio. In certain asymmetric reamplification methods, typically only single-stranded amplicons are generated since the second amplification reaction composition comprises only first or second primers from each primer set or a non-equimolar ratio of first and second primers from a primer set.

In certain embodiments, additional polymerase may also be a component of the second amplification reaction composition. In certain embodiments, there may be sufficient residual polymerase from the first amplification composition to synthesize the second amplification product.

Methods of optimizing amplification reactions are well known to those skilled in the art. For example, it is well known that PCR may be optimized by altering times and temperatures for annealing, polymerization, and denaturing, as well as changing the buffers, salts, and other reagents in the reaction composition. Optimization may also be affected by the design of the amplification primers used. For example, the length of the primers, as well as the G-C:A-T ratio may alter the efficiency of primer annealing, thus altering the amplification reaction. See James G. Wetmur, “Nucleic Acid Hybrids, Formation and Structure,” in Molecular Biology and Biotechnology, pp. 605-8, (Robert A. Meyers ed., 1995).

D. Certain Exemplary Embodiments of Detecting Targets

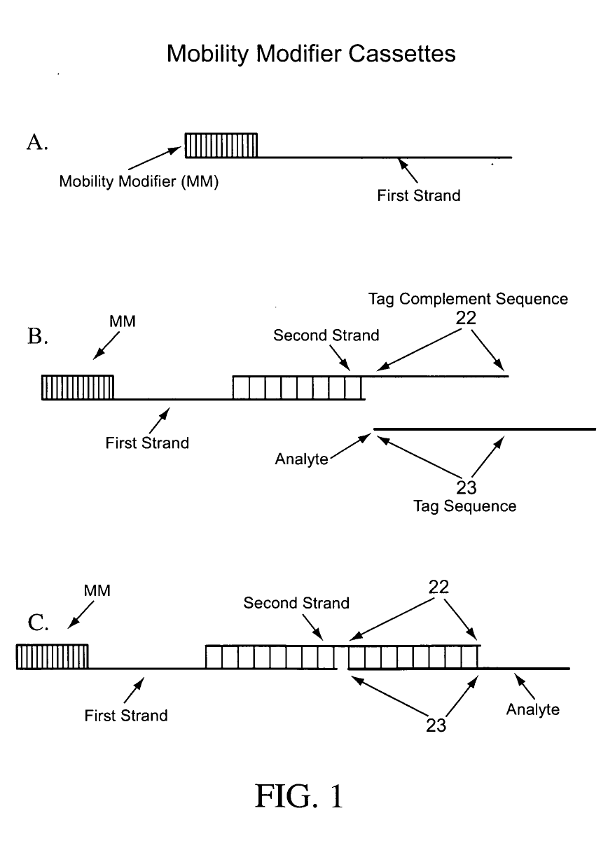

The present invention is directed to methods, mobility cassettes, and kits for detecting analyte polynucleotides that comprise a tag sequence. The mobility modifier cassette comprises a first nucleic acid strand comprising a mobility modifier. See FIG. 1 A. Various mobility modifiers that may be used in certain embodiments are discussed above in detail and include but are not limited to nucleic acids, polymers such as a polyethylene oxide (PEO), polyglycolic acid, polyurethane polymers, polypeptides, or oligosaccharides. Certain mobility modifiers comprising PEO's have been described, e.g., in U.S. Pat. Nos. 5,470,705; 5,580,732; 5,624,800; and 5,989,871.

In certain embodiments, the mobility modifier cassette further comprises a second nucleic acid strand comprising a tag complement sequence that is complementary to the tag sequence of the analyte polynucleotide. See FIG. 1 B. In certain embodiments, a portion of the second nucleic acid strand is hybridized to the first nucleic acid strand and at least a portion of the tag complement sequence 22 is not hybridized to the first nucleic acid strand. In certain embodiments, the tag sequence 23 of the analyte polynucleotide hybridizes to the tag complement sequence 22 of the second strand. See FIG. 1C. In certain embodiments, the first nucleic acid strand is suitable for ligation to the analyte polynucleotide when the second nucleic acid strand and the analyte polynucleotide are hybridized to one another. See FIG. 1 C.

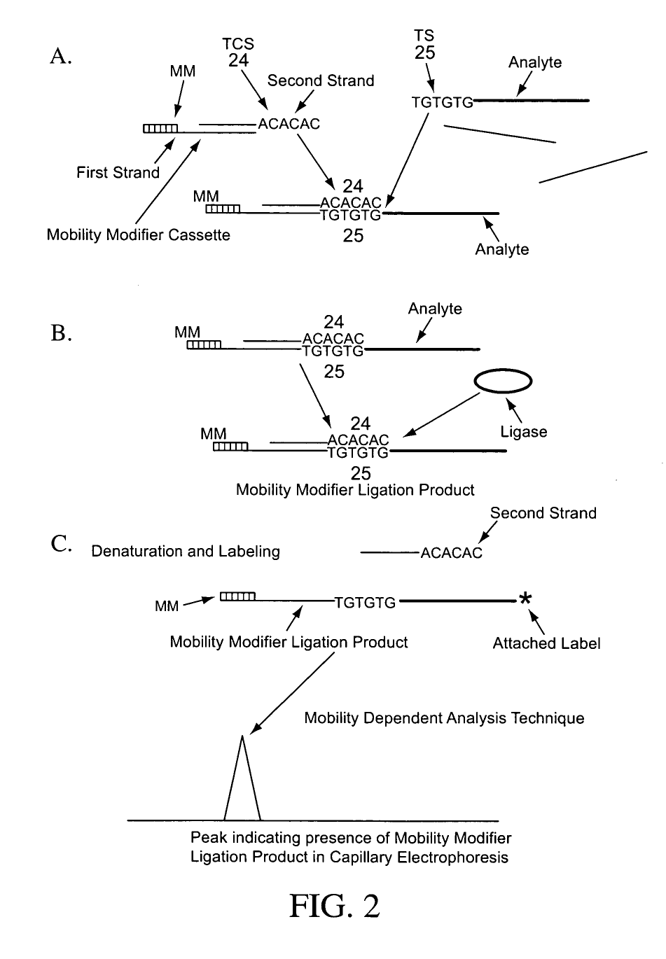

FIG. 2 illustrates certain exemplary embodiments of a method in which one forms a ligation reaction composition comprising at least one mobility modifier cassette and a sample putatively containing an analyte polynucleotide comprising a tag sequence 25 that is complementary to the tag complement sequence 24 of the mobility modifier cassette. FIGS. 2 A and B show exemplary embodiments in which such an analyte polynucleotide is present in the sample, and the tag complement sequence 24 of the mobility modifier cassette hybridizes to the tag sequence 25 of the analyte polynucleotide.

In the exemplary embodiments of FIG. 2, one subjects the ligation reaction composition to at least one cycle of ligation wherein the first nucleic strand of the mobility modifier cassette and the analyte polynucleotide are ligated to form a mobility modifier ligation product comprising the first nucleic acid strand and the analyte polynucleotide. See FIG. 2 B. The second strand of the mobility modifier cassette may or may not be denatured from the first strand. FIG. 2 C illustrates exemplary embodiments where the second strand is denatured from the mobility modifier ligation product, and the mobility modifier ligation product is analyzed using a mobility-dependent analysis technique.

In certain embodiments, the mobility modifier ligation product comprising the analyte polynucleotide and the first strand comprising the mobility modifier may be labeled. As exemplified in FIG. 2 C, in certain embodiments, the label may be attached to the analyte polynucleotide. If the label is attached to the analyte polynucleotide, the label may be attached before, during, or after the analyte polynucleotide is hybridized to the mobility modifier cassette. FIG. 2 C illustrates a label attached to an analyte polynucleotide after the analyte polynucleotide is hybridized to a mobility modifier cassette. In certain embodiments, the label may be attached to the mobility modifier cassette. If the label is attached to the mobility modifier cassette, the label may be attached before, during, or after the analyte polynucleotide is hybridized to the mobility modifier cassette.

In certain embodiments, one can perform multiplex analysis of two or more analyte polynucleotides. In certain embodiments, one may use two or more different mobility modifier cassettes that each have a different mobility to distinguish between two or more different analyte polynucleotides. In certain embodiments, the different analyte polynucleotides may not be easily resolved without the different mobility modifier cassettes that have different mobilities. For example, different analyte polynucleotides may be the same size or be similar in size and may not be easily resolved in a mobility-dependent analysis technique. In certain embodiments, one may desire to determine a particular nucleotide at several different loci in a sample. In certain embodiments, one may employ different probes or primers for the different loci to create different analyte polynucleotides using any of several different types of reactions. In certain instances, the different analyte polynucleotides may not be easily resolved in a mobility-dependent analysis technique. In certain embodiments, different mobility modifier cassettes having different mobilities are used to resolve such analyte polynucleotides.

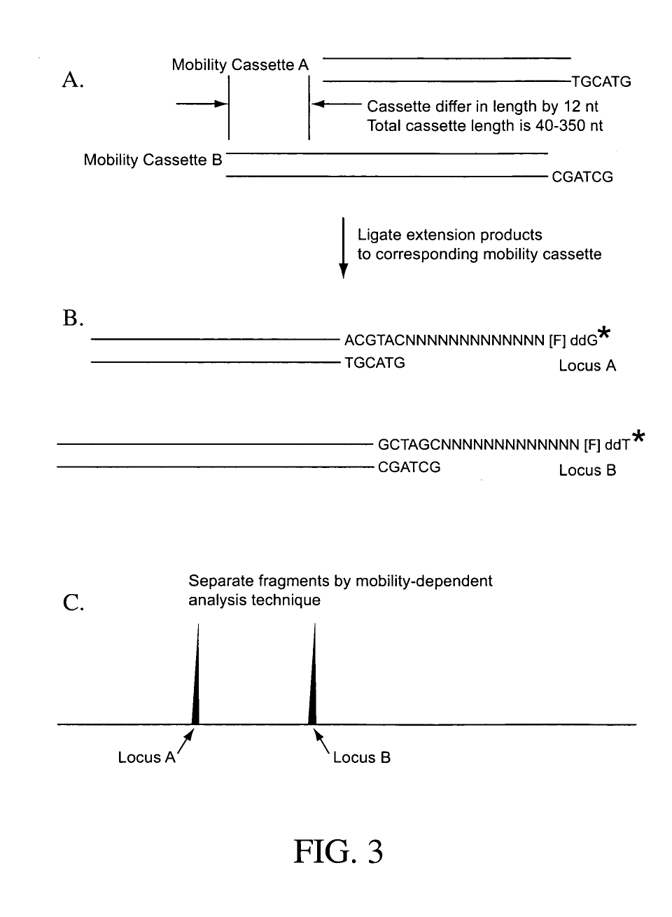

Thus, in certain embodiments, one employs mobility modifier cassettes that have different mobilities in mobility-dependent analysis techniques to distinguish between different analyte polynucleotides that are otherwise not easily distinguished in a mobility-dependent analysis technique. FIG. 3 illustrates certain such embodiments.

FIG. 3 A shows two different mobility modifier cassettes. The first nucleic acid strand of mobility Cassette A is 12 nucleotides shorter than the first nucleic acid strand of Mobility Cassette B. Mobility cassette A further comprises a second nucleic acid strand that comprises a tag complement sequence that is different from the tag complement sequence of the second nucleic acid strand of Mobility Cassette B. The different tag complement sequences hybridize to different analyte polynucleotides. See FIG. 3B. In this example, the two analyte polynucleotides are primer extension products from two different loci designated locus A and locus B. The primer extension products in this example are the same length, and thus, without modification, may not be resolvable using a mobility-dependent analysis technique. The primer extension products in this example are labeled.

After hybridization and ligation of the two primer extension products to the two different mobility cassettes, the primer ligated to Mobility Cassette A is 12 nucleotides shorter than the primer extension product ligated to Mobility Cassette B. See FIG. 3B. Thus, when the two analyte polynucleotides are resolved with a mobility-dependent analysis technique, such as gel electrophoresis or capillary electrophoresis, the two analyte polynucleotides corresponding to the two different loci (A and B) are distinguishable. See the FIG. 3 C.

Various nonlimited examples of methods employing mobility modifier cassettes are now described.

Primer Extension

In certain embodiments, one employs mobility modifier cassettes in a primer extension reaction. In certain such embodiments, one or more primers are extended in a polymerase-mediated primer extension reaction (e.g., Mullis, U.S. Pat. No. 4,683,202; Sanger and Coulson, Proc. Natl. Acad. Sci. USA 74: 5463-5467 (1977)). In certain such embodiments, one carries out primer extension that terminates after a single nucleotide is incorporated into the primer, which results in a primer extension product that includes one more nucleotide than the primer. Such reactions have been called minisequencing, single base extension (SBE), and single nucleotide extension (SNE).

In certain such embodiments, primer extension is carried out in the absence of nucleotides that substantially permit further extension and in the presence of one or more chain-terminating nucleotides, e.g., but not limited to, dideoxynucleotide terminators (e.g., Syvanen et al., Genomics, 8: 684-692 (1990); Soderlund and Syvanen, PCT WO 91/13075; Goelet et al., PCT WO 92/15712). Once a chain-terminating nucleotide is incorporated into a primer extension product, the chain-terminating nucleotide prevents further incorporation of nucleotides to the 3′ end of the primer extension product by polymerase. Thus, the primers are extended by only a single nucleotide, and the identity of that nucleotide provides information about the target sequence immediately adjacent to the 3′-end of the primer. In certain embodiments, different chain-terminating nucleotides are labeled with different labels that may be used to distinguish between the different nucleotides. For example, in certain embodiments, one may use four different labels for each of the different nucleotides A, G, C and T nucleotide, or analogs thereof. In certain embodiments, one may employ four different fluorescent labels.

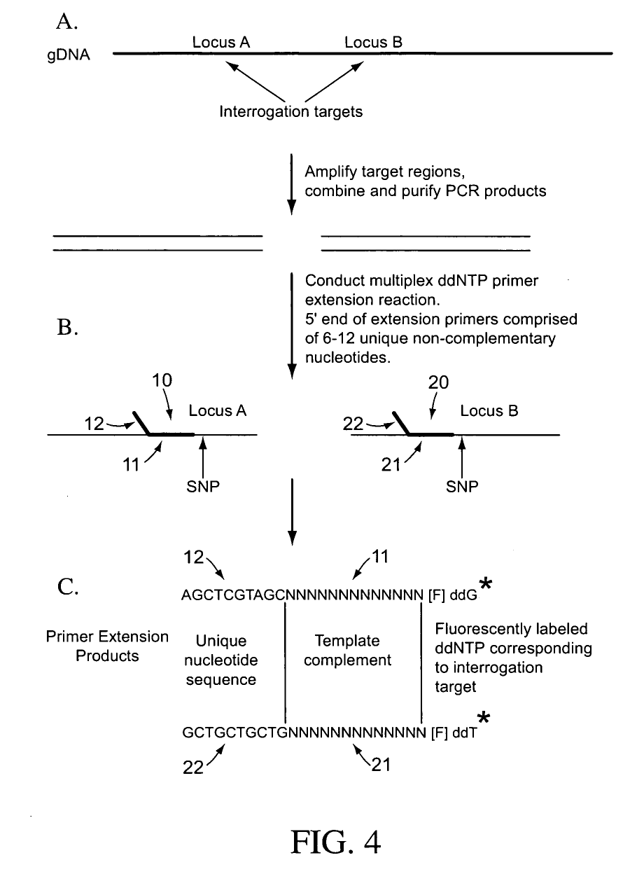

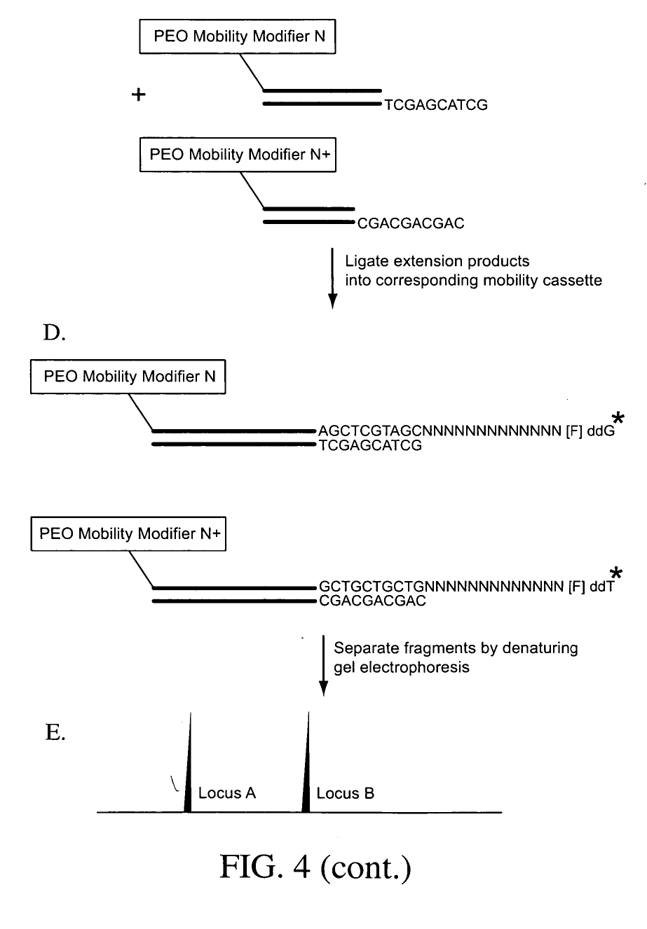

FIG. 4 shows certain nonlimiting embodiments employing mobility modifier cassettes in a primer extension reaction. Specifically, FIG. 4 shows two different targets (Locus A and Locus B) that are amplified by PCR. The PCR products are exposed to two different primers 10 and 20, one for each different locus (see FIG. 4(B)). Each of the primers comprises a different target-specific portion 11 and 21 that is specific for the particular locus and that is complementary to a portion of the target sequence that is immediately 3′ to a single nucleotide polymorphism (SNP) to be detected. Each of the primers 10 and 20 further comprises a different tag sequence 12 and 22. The PCR products are further exposed to polymerase and labeled chain-terminating dideoxyNTP's (ddNTP's). Each of the different ddNTP's is labeled with a different label (e.g., different colors). For example, each ddATP is green, each ddTTP is blue, etc.

The primers and PCR products are then subjected to a polymerase reaction to generate single base primer extension products comprising primers and the labeled dideoxynucleotides (see FIG. 4(C)). The primer extension products are used as analyte polynucleotides.

In the embodiments shown in FIG. 4(C), one forms a ligation reaction composition comprising the primer extension products and two different mobility cassettes. The two different cassettes have different mobility modifiers and have different tag complement sequences (each complementary to a different tag sequence of the two different extension products). The ligation reaction composition is subjected to a ligation reaction to ligate the first strand of the appropriate mobility cassette to the adjacently hybridized primer extension product (see FIG. 4(D)) to form ligation products. The ligation products may then be resolved by electrophoresis or another mobility-dependent analysis technique (see FIG. 4(E)). One determines the identity of the locus by the particular mobility of the ligation product and determines the particular nucleotides at the SNP site of that locus based on the particular label associated with the ligation product.

Oligonucleotide Ligation Assay

In certain embodiments, one employs a ligation reaction that results in a given ligation product if a particular target nucleic acid sequence is present in a sample. See, e.g., those discussed in U.S. Pat. No. 6,027,889, PCT Published Patent Application No. WO 01/92579, and U.S. patent application Ser. Nos. 09/584,905 and 10/011,993. In certain embodiments, such a ligation product or products may be an analyte polynucleotide or analyte polynucleotides, the presence or absence of which are detected with one or more mobility modifier cassettes.

In certain such embodiments, for each target nucleic acid sequence to be detected, one forms a ligation reaction mixture comprising a probe set, comprising at least one first target-specific probe and at least one second target-specific probe, and the sample. In certain embodiments, the at least one first probe comprises a first tag sequence, and the first tag sequence is specific for the first target-specific probe. In certain embodiments, the at least one second probe comprises a label, and the label is specific for the second target-specific probe.

In certain embodiments, the first and second target-specific probes in each probe set are designed to be complementary to the sequences immediately flanking a pivotal nucleotide of a target nucleic acid sequence. In certain embodiments, either the first target-specific probe or the second target-specific probe of a probe set, but not both, will comprise the pivotal complement. When the target nucleic acid sequence is present in the sample, the first and second target-specific probes will hybridize, under appropriate conditions, to adjacent regions on the target. When the pivotal complement is base-paired in the presence of an appropriate ligation agent, two adjacently hybridized probes may be ligated together to form a ligation product.

One can then detect the presence or absence of the target nucleic acid sequences by detecting the presence or absence of the ligation product.

According to certain embodiments, the first and second probes in each ligation probe set are designed to be complementary to the sequences immediately flanking the pivotal nucleotide of the target sequence (see, e.g., probes A, B, and Z in FIG. 11 (A)). In the embodiment shown in FIG. 11, two first probes A and B of a ligation probe set comprise a different nucleotide at the pivotal complement and a different label for each different nucleotide at the pivotal complement. In the embodiment shown in FIG. 11, the second probe Z of the ligation probe set comprises a tag sequence 10 that corresponds to the locus being analyzed. Thus, in certain embodiments, multiple probe sets may be used to detect different alleles at multiple different loci. In certain such embodiments, one may employ multiple probe sets that each include: different first probes that may be used to distinguish different alleles in view of the different labels; and different second probes that may be used to separate analyte polynucleotides for the different loci being analyzed using the different tag sequences and different mobility modifier cassettes that correspond to the tag sequences. One forms a ligation reaction composition comprising the probe set and the sample.

When the target sequence is present in the sample, the first and second probes will hybridize, under appropriate conditions, to adjacent regions on the target (see, e.g., FIG. 11 (B)). When the pivotal complement is base-paired to the target, in the presence of an appropriate ligation agent, two adjacently hybridized probes may be ligated together to form a ligation product (see, e.g., FIG. 11(C)). In certain embodiments, if the pivotal complement of a first probe is not base-paired to the target, no ligation product comprising that mismatched probe will be formed (see, e.g., probe B in FIGS. 11(B) to 11(D). In certain embodiments, ligation of probes with a pivotal complement that is not complementary to the pivotal nucleotide may occur, but such ligation occurs to a measurably lesser extent than ligation of probes with a pivotal complement that is complementary to the pivotal nucleotide.

In the example shown in FIG. 11, a mismatched base at the pivotal nucleotide interferes with ligation, even if both probes are otherwise fully hybridized to their respective target regions. In certain embodiments, other mechanisms may be employed to avoid ligation of probes that do not include the correct complementary nucleotide at the pivotal complement. For example, in certain embodiments, conditions may be employed such that a probe of a ligation probe set will hybridize to the target sequence to a measurably lesser extent if there is a mismatch at the pivotal nucleotide (see, e.g., U.S. Pat. No. 5,521,065). Thus, in such embodiments, such non-hybridized probes will not be ligated to the other probe in the probe set.

In certain embodiments, the first probes and second probes in a ligation probe set are designed with similar melting temperatures (Tₘ). Where a probe includes a pivotal complement, in certain embodiments, the Tₘ for the probe(s) comprising the pivotal complement(s) of the target pivotal nucleotide sought will be approximately 4-15° C. lower than the other probe(s) that do not contain the pivotal complement in the probe set. In certain such embodiments, the probe comprising the pivotal complement(s) will also be designed with a Tₘ near the ligation temperature. Thus, a probe with a mismatched nucleotide will more readily dissociate from the target at the ligation temperature. The ligation temperature, therefore, in certain embodiments provides another way to discriminate between, for example, multiple potential alleles in the target.

Further, in certain embodiments, ligation probe sets do not comprise a pivotal complement at the terminus of the first or the second probe (e.g., at the 3′ end or the 5′ end of the first or second probe). Rather, the pivotal complement is located somewhere between the 5′ end and the 3′ end of the first or second probe. In certain such embodiments, probes with target-specific portions that are fully complementary with their respective target regions will hybridize under high stringency conditions. Probes with one or more mismatched bases in the target-specific portion, by contrast, will hybridize to their respective target region to a measurably lesser extent. Both the first probe and the second probe must be hybridized to the target for a ligation product to be generated.

In certain embodiments as shown in FIG. 11(E), after ligation of appropriately hybridized first and second probes, the ligation products are exposed to mobility modifier cassettes. Mobility modifier cassettes that include a tag complement sequence 11 that is complementary to tag sequences 10 of ligation products hybridize to such ligation products. In the embodiments shown in FIG. 11, the first strand of appropriately hybridized mobility modifier cassettes are ligated to the ligation products. One can then separate out the ligation products using the mobility modifiers and can determine the pivotal nucleotide of the target nucleic acid sequence in view of the label.

FIG. 9 shows certain embodiments in which one employs a probe set that is similar to the probe set shown in FIG. 11, but in which the two first probes A and B of a ligation probe set comprise a different nucleotide at the pivotal complement and a different tag sequence (12 or 14) for each different nucleotide at the pivotal complement. In the embodiment shown in FIG. 9, the second probe Z of the ligation probe set comprises a different label that corresponds to the locus being analyzed. Thus, in certain embodiments, multiple probe sets may be used to detect different alleles at multiple different loci. In certain such embodiments, one may employ multiple probe sets that each include: different first probes that may be used to separate analyte polynucleotides for the different alleles using the different tag sequences and different mobility modifier cassettes that correspond to the tag sequences; and different second probes that may be used to distinguish between different loci being analyzed in view of the different labels.

In certain embodiments, one may subject the initial sample to an amplification reaction to increase the amount of target nucleic acid to which probes in a probe set will hybridize.

In certain embodiments, including, but not limited to, detecting multiple alleles, the ligation reaction mixture may comprise a different probe set for each potential allele in a multiallelic target locus. In certain embodiments, one may use, for example, without limitation, a screening assay to detect the presence of three biallelic loci (e.g., L1, L2, and L3) in an individual using six probe sets. See, e.g., Table 1 below.

TABLE 1

| Locus | Allele | Probe Set -- Probe |
|---|---|---|
| L1 | 1 | A (red) -- Z (tag 1) |
| | 2 | B (blue) -- Z (tag 1) |
| L2 | 1 | C (red) -- Y (tag 2) |
| | 2 | D (blue) -- Y (tag 2) |
| L3 | 1 | E (red) -- X (tag 3) |
| | 2 | F (blue) -- X (tag 3) |

In such embodiments, two different probe sets are used to detect the presence or absence of each allele at each locus. The two first target-specific probes of the two different probe sets for each locus, for example, probes A and B for locus L1, comprise the same upstream sequence-specific portion, but differ at the pivotal complement. Also, the two different probes A and B comprise different labels. The two second target-specific probes of the two different probe sets for each locus, for example, probe Z for locus L1, comprise the same downstream sequence-specific portion. Also, the probes Z comprise the same tag sequence. The tag sequence of the second target-specific probe for each different locus is different. Thus, each different tag sequence may hybridize to a different tag complement of a different mobility modifier cassette. Accordingly, ligation products for each different loci may be ligated to a different mobility modifier cassette. Both allelic combinations of a probe set, such as AZ and BZ of the probe set for locus L1, will have the same mobility, and thus both alleles may be detected at the same position after a mobility dependent analysis technique. Therefore, the label for alleles of locus L1 will be detected at position 1, the labels for alleles for locus L2 will be detected at position 2, and the labels for alleles of locus L3 will be detected at position 3.

Thus, in embodiments as depicted in Table 1, three probes A, B, and Z, are used to form the two possible L1 ligation products, wherein AZ is the ligation product of the first L1 allele and BZ is the ligation product of the second L1 allele. Likewise, probes C, D, and Y, are used to form the two possible L2 ligation products. Likewise, probes E, F, and X, are used to form the two possible L3 ligation products.

After ligation of adjacently hybridized first and second target-specific probes, one can detect the presence or absence of a ligation product for each of the alleles for each of the loci by detecting the presence or absence of the unique combinations of labels and mobility for each allele. For example, one may detect the allele for L1 at the mobility conferred by the mobility modifier of the mobility modifier cassette comprising tag 1 (position 1). For example, one individual may have a red label at position 1, a blue label at position 2, and both red and blue labels at position 3. Such an individual would be determined to be homozygous for allele 1 at locus L1, homozygous for allele 2 at locus L2, and heterozygous for both alleles 1 and 2 at locus L3.

The person of ordinary skill will appreciate that in certain embodiments, three or more alleles at a multiallelic locus can also be differentiated using these methods. Also, in certain embodiments, more than one loci can be analyzed.

The skilled artisan will understand that in certain embodiments, the probes can be designed with the pivotal complement at any location in either the first target-specific probe or the second target-specific probe. Additionally, in certain embodiments, target-specific probes comprise multiple pivotal complements.

Oligonucleotide Ligation and Amplification

In certain embodiments, one employs a ligation reaction followed by amplification to obtain analyte polynucleotides to detect target nucleic acids. Certain nonlimiting examples are shown in FIGS. 7 and 10.

According to certain embodiments, the first and second probes in each ligation probe set are designed to be complementary to the sequences immediately flanking the pivotal nucleotide of the target sequence (see, e.g., probes A, B, and Z in FIG. 10(A)). In the embodiment shown in FIG. 10, two first probes A and B of a ligation probe set comprise a different nucleotide at the pivotal complement and a different 5′ primer-specific portion (A1 or B1) for each different nucleotide at the pivotal complement. In the embodiment shown in FIG. 10, the second probe Z of the ligation probe set comprises a tag sequence 16 that corresponds to the locus being analyzed and a 3′ primer-specific portion Z1. Thus, in certain embodiments, multiple probe sets may be used to detect different alleles at multiple different loci. In certain such embodiments, one may employ multiple probe sets that each include: different first probes that may be used to distinguish different alleles in view of the different 5′ primer-specific portions; and different second probes that may be used to separate analyte polynucleotides for the different loci being analyzed using the different tag sequences and different mobility modifier cassettes that correspond to the tag sequences. In certain embodiments, one forms a ligation reaction composition comprising the probe set and the sample.

When the target sequence is present in the sample, the first and second probes will hybridize, under appropriate conditions, to adjacent regions on the target (see, e.g., FIG. 10(B)). When the pivotal complement is base-paired to the target, in the presence of an appropriate ligation agent, two adjacently hybridized probes may be ligated together to form a ligation product (see, e.g., FIG. 10(C)). In certain embodiments, if the pivotal complement of a first probe is not base-paired to the target, no ligation product comprising that mismatched probe will be formed (see, e.g., probe B in FIGS. 10(B) to 10(D). In certain embodiments, ligation of probes with a pivotal complement that is not complementary to the pivotal nucleotide may occur, but such ligation occurs to a measurably lesser extent than ligation of probes with a pivotal complement that is complementary to the pivotal nucleotide.

In the example shown in FIGS. 10 B and C, probe B contains a mismatched base at the pivotal nucleotide. This mismatched base at the pivotal nucleotide interferes with ligation, even if both probes are otherwise fully hybridized to their respective target regions. In certain embodiments, other mechanisms may be employed to avoid ligation of probes that do not include the correct complementary nucleotide at the pivotal complement. For example, in certain embodiments, conditions may be employed such that a probe of a ligation probe set will hybridize to the target sequence to a measurably lesser extent if there is a mismatch at the pivotal nucleotide. Thus, in such embodiments, such non-hybridized probes will not be ligated to the other probe in the probe set.

In certain embodiments, the first probes and second probes in a ligation probe set are designed with similar melting temperatures (Tₘ). Where a probe includes a pivotal complement, in certain embodiments, the Tₘ for the probe(s) comprising the pivotal complement(s) of the target pivotal nucleotide sought will be approximately 4-15° C. lower than the other probe(s) that do not contain the pivotal complement in the probe set. In certain such embodiments, the probe comprising the pivotal complement(s) will also be designed with a Tₘ near the ligation temperature. Thus, a probe with a mismatched nucleotide will more readily dissociate from the target at the ligation temperature. The ligation temperature, therefore, in certain embodiments provides another way to discriminate between, for example, multiple potential alleles in the target.

Further, in certain embodiments, ligation probe sets do not comprise a pivotal complement at the terminus of the first or the second probe (e.g., at the 3′ end or the 5′ end of the first or second probe). Rather, the pivotal complement is located somewhere between the 5′ end and the 3′ end of the first or second probe. In certain such embodiments, probes with target-specific portions that are fully complementary with their respective target regions will hybridize under high stringency conditions. Probes with one or more mismatched bases in the target-specific portion, by contrast, will hybridize to their respective target region to a measurably lesser extent. Both the first probe and the second probe must be hybridized to the target for a ligation product to be generated.

The ligation reaction composition (in the appropriate salts, buffers, and nucleotide triphosphates) is then combined with at least one primer set and a polymerase to form a first amplification reaction composition (see, e.g., FIG. 10(E)). In the first amplification cycle, the second primer, comprising a sequence complementary to the 3′ primer-specific portion of the ligation product, hybridizes with the ligation product and is extended in a template-dependent fashion to create a double-stranded molecule comprising the ligation product and its complement (see, e.g., FIG. 10(E)-(F)). Subsequent amplification cycles may exponentially amplify this double-stranded molecule (see, e.g., FIG. 10(F)-(J)). In FIG. 10, for example, primers PA* and PB* include different labels. Thus, amplification products resulting from incorporation of these primers will include a label specific for the particular pivotal nucleotide that is included in the original target sequence.

In the embodiments shown in FIG. 10(K), following amplification, the amplification products are exposed to mobility modifier cassettes. The mobility modifier cassettes include both a tag complement sequence 17 that is complementary to tag sequence 16 of the amplification products and an adjacent sequence PZ1 that is complementary to the 3′ primer-specific portion sequence Z1. In certain embodiments shown in FIG. 10, the mobility modifier cassettes hybridize to the appropriate amplification products. In the embodiments shown in FIG. 10, the first strand of appropriately hybridized mobility modifier cassettes are ligated to the amplification products. One can then separate out the amplification products using the mobility modifiers and can determine the pivotal nucleotide of the target nucleic acid sequence in view of the label.

FIG. 8 shows certain embodiments in which one employs a probe set that is similar to the probe set shown in FIG. 10, but in which the two first probes A and B of a ligation probe set comprise a 5′ primer-specific portion (PSP), a different nucleotide at the pivotal complement, and a different tag sequence (18 or 20) for each different nucleotide at the pivotal complement. In the embodiment shown in FIG. 8, the second probe Z of the ligation probe set comprises a 3′ primer-specific portion Z1 that corresponds to the locus being analyzed. Thus, in certain embodiments, multiple probe sets may be used to detect different alleles at multiple different loci. In certain such embodiments, one may employ multiple probe sets that each include: different first probes that may be used to separate analyte polynucleotides for different alleles using the different tag sequences and different mobility modifier cassettes that correspond to the tag sequences; and different second probes that may be used to distinguish analyte polynucleotides for the different loci in view of the different 3′ primer-specific portions. One forms a ligation reaction composition comprising the probe set and the sample.

FIGS. 8(A) to (K) illustrate certain embodiments employing such probe sets and mobility modifier cassettes.

Tagman Assays

In certain embodiments, the analytepolynucleotide is a fluorescently labeled product from a Taqman™ assay. The Taqman™ probes and procedures for using them are described in, e.g., U.S. Pat. No. 5,538,848. The Taqman™ assay utilizes the 5′-nuclease activity of a DNA polymerase. A Taqman™ probe hybridizes to a target nucleic acid sequence if the target is present. The Taqman™ probe comprises a fluorescent molecule on one end of the probe, and a quenching molecule at the other end of the probe. When the probe is intact with both the fluorescent molecule and quenching molecule attached, there is little to no fluorescence.

Primers are added that also hybridize to the target nucleic acid sequence. A polymerization reaction is then started at the primer, which adds nucleotides to the end of the primer. The Taqman™ probe on the target nucleic acid sequence is cleaved during the polymerization reaction as a result of the strand replacement that occurs during DNA polymerization. That cleavage separates the fluorescent molecule from the quenching molecule on the probe, which results in an increase in detectable fluorescence from the fluorescent molecule. Thus, the detection of fluorescence indicates the presence of the particular target nucleic acid sequence involved in the polymerase reaction.

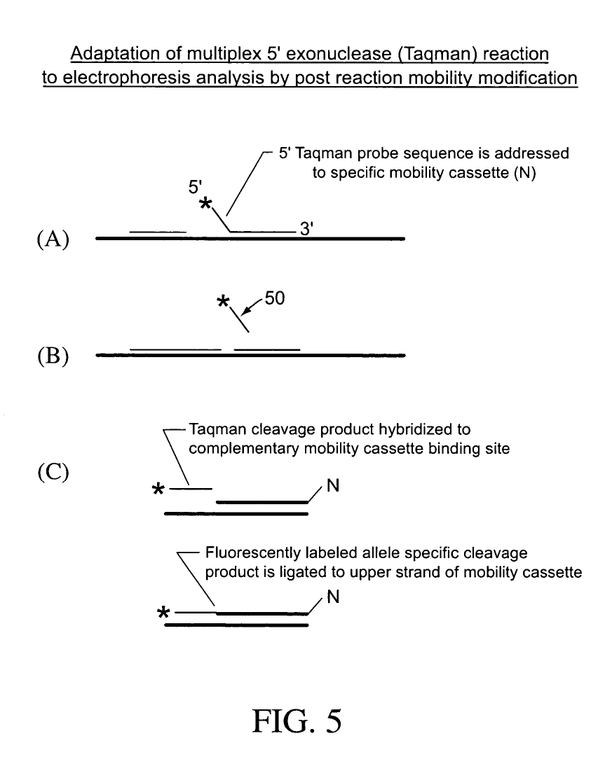

FIG. 5 illustrates certain embodiments employing mobility modifier cassettes and Taqman™ probes. FIG. 5 shows a Taqman™ probe comprising a label attached to a 5′ portion of the probe, and a 3′ portion of the probe that is complementary to the target to be detected. The 5′ portion of the probe includes a tag sequence that does not complement the target. When in the presence of a complementary target nucleic acid sequence, the Taqman™ probe hybridizes to the target (FIG. 5(A)), and is cleaved by a polymerase reaction (FIG. 5(B)), forming a cleavage product 50 that comprises the label and the tag sequence.

A mobility modifier cassette is then added to the sample containing the cleavage product 50. See, e.g., FIG. 5(C). The mobility modifier cassette comprises a first strand attached to a mobility modifier, and a second strand comprising a portion that is complementary to the first strand, and a portion that comprises a tag sequence complement that does not hybridize to the first strand. The tag sequence complement is complementary to the tag sequence of the cleavage product 50.

FIG. 5 shows the cleavage product 50 hybridized to the mobility cassette, and ligated to the first strand of the mobility modifier cassette. The ligation product that is formed may then be resolved by a mobility dependent analysis technique.

Cleavage Assays

In certain embodiments, the analyte polynucleotide is the cleavage product from a cleavage assay such as an Invader™ assay. Nucleic acids in a sample may be subjected to a cleavage procedure such as the cleavage procedure in an Invader™ assay (as exemplified, e.g., in U.S. Pat. Nos. 5,846,717; 5,985,557; 5,994,069; 6,001,567; and 6,090,543). Such procedures produce a cleavage product when a nucleic acid of interest is present in a sample.

In certain embodiments, the analyte polynucleotide may be such a cleavage product. Briefly, the cleavage procedure may employ two nucleic acid oligonucleotides that are designed to be complementary to the nucleic acid sequence in the sample. A first oligonucleotide comprises a 5′ portion that does not complement the nucleic acid in the sample that is contiguous with a 3′ portion that does complement the nucleic acid in the sample. A second oligonucleotide complements the nucleic acid in the sample in a region of the nucleic acid in the sample that is 3′ of the region complemented by the first oligonucleotide, and includes a complementary portion that slightly overlaps with the region complemented by the first oligonucleotide. Hybridization of the two oligonucleotides to the nucleic acid in the sample causes a portion of the first oligonucleotide to be cleaved, often in the presence of an enzyme such as a flap endonuclease (FEN). Cleavage enzymes such as flap endonuclease (FEN) have been described, e.g., in U.S. Pat. Nos. 5,614,402 and 5,795,763. The cleavage product is typically the 5′ portion of the first oligonucleotide that does not complement the nucleic acid in the sample, and that portion of the complementary region that overlaps with the second oligonucleotide. This cleavage product comprises a known nucleic acid sequence. In certain embodiments, such cleavage products may be analytes.

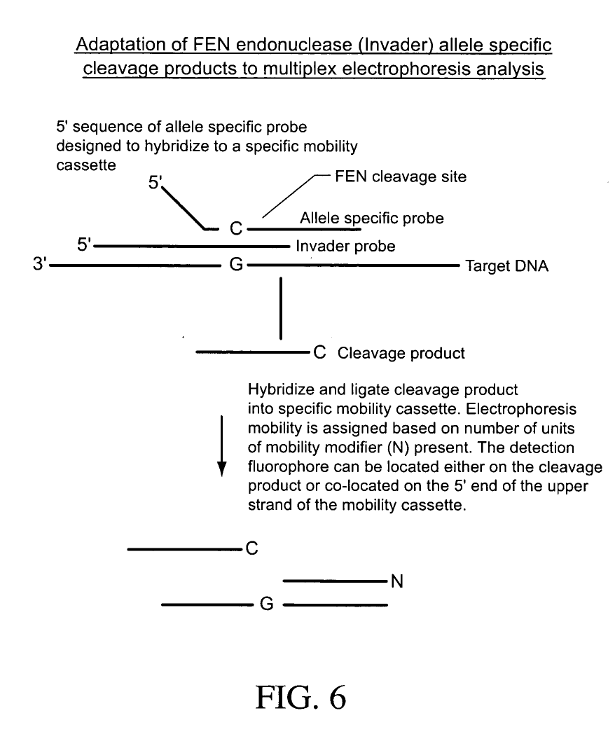

As a non-limiting example, FIG. 6 shows detection of a target nucleic acid sequence using an allele-specific probe. The allele-specific probe in FIG. 6 can be used to detect an allele that differs from other alleles by a single nucleotide. In FIG. 6, the allele-specific probe comprises (a) a 5′ portion that is not complementary to the target nucleic acid sequence and that comprises a tag sequence, and (b) an allele-specific portion that is complementary to the target nucleic acid sequence. A second probe (an invader probe) is used that also complements the target polynucleotide and overlaps with the allele-specific probe.

In the embodiments shown in FIG. 6, an endonuclease (such as FEN) is added that cleaves the portion of the allele-specific probe that overlaps with the invader probe to form a cleavage product. The cleavage product comprises the 5′ portion of the allele-specific probe that does not complement the target polynucleotide and that comprises the tag sequence.

In the embodiments shown in FIG. 6, the cleavage product is exposed to a mobility modifier cassette. The mobility modifier cassette comprises a first strand attached to a mobility modifier, and a second strand comprising a portion that is complementary to the first strand, and a portion that comprises a tag sequence complement that does not hybridize to the first strand. The tag sequence complement is complementary to the tag sequence of the cleavage product.

The cleavage product hybridizes to the mobility modifier cassette, and a ligation reaction is performed to ligate the cleavage product to the mobility cassette. The ligation product may then be detected by a mobility-dependent analysis technique.

In certain embodiments, a label is attached to the cleavage product. In certain embodiments, a label is attached to the first strand of the mobility modifier cassette.

1. A method of detecting at least one analyte polynucleotide comprising a tag sequence, the method comprising:
(a) forming a ligation reaction composition comprising the at least one analyte polynucleotide and at least one mobility cassette, wherein the at least one mobility cassette comprises:
a first nucleic acid strand comprising a mobility modifier; and
a second nucleic acid strand comprising a tag complement sequence that is complementary to the tag sequence of the analyte polynucleotide, wherein a portion of the second nucleic acid strand is hybridized to the first nucleic acid strand and wherein at least a portion of the tag complement sequence is not hybridized to the first nucleic acid strand, and
wherein the first nucleic acid strand is suitable for ligation to the analyte polynucleotide when the first nucleic acid strand and the analyte polynucleotide are hybridized adjacent to one another on the second nucleic acid strand;
(b) subjecting the ligation reaction composition to at least one cycle of ligation, wherein the first nucleic acid strand and the analyte polynucleotide are ligated to form a mobility modifier ligation product comprising the first nucleic acid strand and the analyte polynucleotide; and
(c) detecting the at least one analyte polynucleotide by analyzing the mobility modifier ligation product using a mobility-dependent analysis technique.

2. A method of detecting at least one primer extension product comprising:
(a) forming an extension reaction composition comprising a polymerase, at least one target nucleic acid sequence, and a primer for each target nucleic acid sequence, the primer comprising a target-specific portion and a tag sequence;
(b) incubating the extension reaction composition under appropriate conditions to generate a primer extension product for each target nucleic acid, the primer extension product comprising the tag sequence;
(c) forming a ligation reaction composition comprising the primer extension product for each target nucleic acid and a mobility cassette for each target nucleic acid, wherein the mobility cassette comprises:
a first nucleic acid strand comprising a mobility modifier; and
a second nucleic acid strand comprising a complementary tag sequence that is complementary to the tag sequence of the primer extension product, wherein a portion of the second nucleic acid strand is hybridized to the first nucleic acid strand and wherein at least a portion of the complementary tag sequence is not hybridized to the first nucleic acid strand, and
wherein the first nucleic acid strand is suitable for ligation to the primer extension product when the first nucleic acid strand and the primer extension product are hybridized adjacent to one another on the second nucleic acid strand;
(d) subjecting the ligation reaction composition to at least one cycle of ligation, wherein the first nucleic acid strand and the primer extension product for each target nucleic acid that are adjacently hybridized on the second nucleic acid strand for each target nucleic acid are ligated to form a mobility modifier ligation product comprising the first nucleic acid strand and the primer extension product; and
(e) detecting the primer extension product for each target nucleic acid sequence by analyzing the mobility modifier ligation product or a portion of the mobility modifier ligation product for each primer extension product using a mobility-dependent analysis technique.

3. A method of detecting at least one target nucleic acid sequence in a sample comprising:
(a) forming a first ligation reaction composition comprising the sample and a probe set for each target nucleic acid sequence, the probe set comprising (i) at least one first probe, comprising a first target-specific portion, and (ii) at least one second probe, comprising a second target-specific portion, wherein the probes in each set are suitable for ligation together when hybridized adjacent to one another on the at least one target nucleic acid sequence, and wherein at least one probe in each probe set further comprises at least one tag sequence;
(b) subjecting the first ligation reaction composition to at least one cycle of ligation, wherein adjacently hybridized probes are ligated to form a first ligation product comprising the first and second target-specific portions and the tag sequence;
(c) forming a second ligation reaction composition comprising the first ligation product and a mobility cassette, wherein the mobility cassette comprises:
a first nucleic acid strand comprising a mobility modifier; and
a second nucleic acid strand comprising a complementary tag sequence that is complementary to the tag sequence of the first ligation product, wherein a portion of the second nucleic acid strand is hybridized to the first nucleic acid strand and wherein at least a portion of the complementary tag sequence is not hybridized to the first nucleic acid strand, and
wherein the first nucleic acid strand is suitable for ligation to the first ligation product when the first nucleic acid strand and the first ligation product are hybridized adjacent to one another on the second nucleic acid strand;
(d) subjecting the second ligation reaction composition to at least one cycle of ligation, wherein the first nucleic acid strand and the first ligation product that are adjacently hybridized on the second nucleic acid strand are ligated to form a mobility modifier ligation product comprising the first nucleic acid strand and the first ligation product; and
(e) detecting the at least one target nucleic acid sequence by analyzing the mobility modifier ligation product or a portion of the mobility modifier ligation product using a mobility-dependent analysis technique.

4. A method for detecting at least one target nucleic acid sequence in a sample comprising:
(a) forming a first ligation reaction composition comprising: the sample and a probe set for each target nucleic acid sequence, the probe set comprising (a) at least one first probe, comprising a first target-specific portion, a tag sequence, and a 5′ primer-specific portion, wherein the 5′ primer-specific portion comprises a sequence, and wherein the tag sequence is located between the 5′ primer-specific portion and the first target-specific portion and (b) at least one second probe, comprising a second target-specific portion and a 3′ primer-specific portion, wherein the 3′ primer-specific portion comprises a sequence, wherein the probes in each set are suitable for ligation together when hybridized adjacent to one another on the at least one target nucleic acid sequence;
(b) subjecting the ligation reaction composition to at least one cycle of ligation, wherein adjacently hybridized probes are ligated to form a first ligation product comprising the first and second target-specific portions, the tag sequence, and the 3′ and 5′ primer-specific portions;
(c) forming a first amplification reaction comprising a DNA polymerase, the first ligation product, and at least one primer set comprising at least one first primer comprising the sequence of the 5′ primer-specific portion, and at least one second primer comprising a sequence complementary to the sequence of the 3′ primer-specific portion;
(d) subjecting the first amplification reaction composition to at least one cycle of amplification to generate an amplification product;
(e) forming a second ligation reaction composition comprising the amplification product and a mobility cassette, wherein the mobility cassette comprises:
a first nucleic acid strand comprising a mobility modifier; and
a second nucleic acid strand comprising a complementary tag sequence that is complementary to the 5′ primer-specific portion and the tag sequence of the amplification product, wherein a portion of the second nucleic acid strand is hybridized to the first nucleic acid strand and wherein at least a portion of the complementary tag sequence is not hybridized to the first nucleic acid strand, and
wherein the first nucleic acid strand is suitable for ligation to the amplification product when the first nucleic acid strand and the amplification product are hybridized adjacent to one another on the second nucleic acid strand;
(f) subjecting the second ligation reaction composition to at least one cycle of ligation, wherein the first nucleic acid strand and the amplification product that are adjacently hybridized on the second nucleic acid strand are ligated to form a mobility modifier ligation product comprising the first nucleic acid strand and the amplification product; and
(g) detecting the at least one target nucleic acid sequence by analyzing the mobility modifier ligation product or a portion of the mobility modifier ligation product using a mobility-dependent analysis technique.

5. A method for detecting at least one target nucleic acid sequence in a sample comprising:
(a) forming a first ligation reaction composition comprising: the sample and a probe set for each target nucleic acid sequence, the probe set comprising (a) at least one first probe, comprising a first target-specific portion and a 5′ primer-specific portion, wherein the 5′ primer-specific portion comprises a sequence, and (b) at least one second probe, comprising a second target-specific portion, a tag sequence, and a 3′ primer-specific portion, wherein the 3′ primer-specific portion comprises a sequence, and wherein the tag sequence is located between the 3′ primer-specific portion and the second target-specific portion, wherein the probes in each set are suitable for ligation together when hybridized adjacent to one another on the at least one target nucleic acid sequence;
(b) subjecting the first ligation reaction composition to at least one cycle of ligation, wherein adjacently hybridized probes are ligated to form a first ligation product comprising the first and second target-specific portions, the tag sequence, and the 3′ and 5′ primer-specific portions;
(c) forming a first amplification reaction composition comprising a DNA polymerase, the first ligation product, and at least one primer set comprising at least one first primer comprising the sequence of the 5′ primer specific portion, and at least one second primer comprising a sequence complementary to the sequence of the 3′ primer-specific portion;
(d) subjecting the first amplification reaction composition to at least one cycle of amplification to generate an amplification product;
(e) forming a second ligation reaction composition comprising the amplification product and a mobility cassette, wherein the mobility cassette comprises:
a first nucleic acid strand comprising a mobility modifier; and
a second nucleic acid strand comprising a complementary tag sequence that is complementary to the 3′ primer-specific portion and the tag sequence of the amplification product, wherein a portion of the second nucleic acid strand is hybridized to the first nucleic acid strand and wherein at least a portion of the complementary tag sequence is not hybridized to the first nucleic acid strand, and
wherein the first nucleic acid strand is suitable for ligation to the amplification product when the first nucleic acid strand and the amplification product are hybridized adjacent to one another on the second nucleic acid strand;
(f) subjecting the second ligation reaction composition to at least one cycle of ligation, wherein the first nucleic acid strand and the amplification product that are adjacently hybridized on the second nucleic acid strand are ligated to form a mobility modifier ligation product comprising the first nucleic acid strand and the amplification product; and
(g) detecting the at least one target nucleic acid sequence by analyzing the mobility modifier ligation product or a portion of the mobility modifier ligation product using a mobility-dependent analysis technique.

6. A mobility cassette comprising:
a first nucleic acid strand comprising a mobility modifier; and
a second nucleic acid strand comprising a complementary tag sequence that is complementary to a tag sequence of a target product, wherein a portion of the second nucleic acid strand is hybridized to the first nucleic acid strand and wherein at least a portion of the complementary tag sequence is not hybridized to the first nucleic acid strand, and
wherein the first nucleic acid strand is suitable for ligation to the target product when the first nucleic acid strand and the target product are hybridized adjacent to one another on the second nucleic acid strand.

7. The mobility cassette of claim 6, wherein the mobility modifier comprises a polyethylene oxide molecule.

8. The mobility cassette of claim 6, wherein the mobility modifier is a polynucleotide.

9. A kit for detecting at least one target nucleic acid sequence comprising:
(a) at least one of (i) a probe comprising a tag sequence and a target-specific portion that can hybridize to a target nucleic acid sequence; and (ii) a primer comprising a tag sequence and a target-specific portion that can hybridize to a target nucleic acid sequence;
(b) a mobility cassette comprising;
a first nucleic acid strand comprising a mobility modifier; and
a second nucleic acid strand comprising a complementary tag sequence that is complementary to the tag sequence, wherein a portion of the second nucleic acid strand is hybridized to the first nucleic acid strand and wherein at least a portion of the complementary tag sequence is not hybridized to the first nucleic acid strand, and
wherein the first nucleic acid strand is suitable for ligation to the tag sequence when the first nucleic acid strand and the tag sequence are hybridized adjacent to one another on the second nucleic acid strand.


SEQUENCE LISTING (6 sequences)

SEQ ID NO: 1 - DNA, 20 bp, Artificial Sequence
  Hypothetical primer extension product
acgtacnnnnnnnnnnnnng

SEQ ID NO: 2 - DNA, 20 bp, Artificial Sequence
  Hypothetical primer extension product
gctagcnnnnnnnnnnnnnt

SEQ ID NO: 3 - DNA, 24 bp, Artificial Sequence
  Hypothetical primer extension product
agctcgtagcnnnnnnnnnnnnng

SEQ ID NO: 4 - DNA, 24 bp, Artificial Sequence
  Hypothetical primer extension product
gctgctgctgnnnnnnnnnnnnnt

SEQ ID NO: 5 - DNA, 10 bp, Artificial Sequence
  Hypothetical tag complement sequence
gctacgagct

SEQ ID NO: 6 - DNA, 10 bp, Artificial Sequence
  Hypothetical tag complement sequence
cagcagcagc